(19) United States
(12) Patent Application Publication
(10) Pub. No.: US 2006/0247244 A1
(43) Pub. Date: Nov. 2, 2006

(54) Quinoxalinones and their use especially in the treatment of cardiovascular diseases

(75) Inventors: Jens-Kerim Ergüden, LEVERKUSEN (DE); Peter Kolkhof, Wuppertal (DE); Julio Castro-Palomino, Barcelona (ES); Alexander Kuhl, Hagen (DE); Raimund Kast, Wuppertal (DE); Johannes-Peter Stasch, Solingen (DE); Hanna Tinel, Wuppertal (DE); Klaus Münter, Wulfrath (DE); Klemens Lustig, Wuppertal (DE); Josef Pernerstorfer, Hilden (DE); Martin Bechem, Wuppertal (DE); Jörg Hüser, Wuppertal (DE)

Correspondence Address: JEFFREY M. GREENMAN, BAYER PHARMACEUTICALS CORPORATION, 400 MORGAN LANE, WEST HAVEN, CT, 06516, US

(73) Assignee: BAYER HEALTHCARE AG, LEVERKUSEN (DE)

(21) Appl. No.: 10/503,521

(22) PCT Filed: Jan. 27, 2003
(86) PCT No.: PCT/EP03/00782

§ 371 Date: Jun. 13, 2006

(30) Foreign Application Priority Data
Feb. 8, 2002 (DE) 102 02 219.0

(51) Int. Cl. A61K 31/498; C07D 241/36

(52) U.S. Cl. 514/249; 544/354

(57) ABSTRACT

The invention relates to quinoxalinones and to methods for producing the same as well as the use thereof for preparing drugs for the treatment and/or prophylaxis of diseases, especially of cardiovascular diseases.

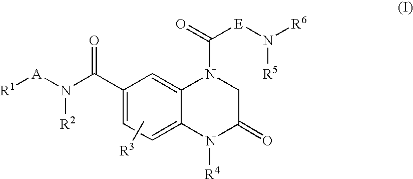

The invention relates to quinoxalinones, to a process for their preparation and to their use for producing medicaments for the treatment and/or prophylaxis of diseases, especially of cardiovascular disorders.

Acetylcholine is the transmitter of the parasympathetic nervous system. This part of the autonomic nervous system has a crucial influence on fundamental processes of a wide variety of organ functions, such as, for example, lung, bladder, stomach and intestine, glands, brain, eye, blood vessels and heart.

Acetylcholine itself cannot be used therapeutically because it is very rapidly inactivated by acetylcholin esterase, but its effect can be imitated by direct parasympathomimetic agents such as, for example, carbachol. Active substances which have an agonistic effect like acetylcholine on the muscarinic (M) acetylcholine receptors may thus influence and control numerous functions depending on the organ or tissue system. For example, activation of muscarinic acetylcholine receptors in the brain may influence the memory and learning processes and pain processing.

It is possible, for example, by using receptor subtype-specific agonists to reduce via the muscarinic M2 acetylcholine receptor, which is expressed particularly strongly in myocardial cells, the heart rate and the contractility after beta-adrenergic stimulation (B. Rauch, F. Niroomand, J. Eur. Heart. 1991, 12, 76-82). Both effects reduce the myocardial oxygen consumption.

Substances of similar structure to the compounds of the invention are known in other indications and for other mechanisms of action. Thus, for example, WO 00/00478, EP-A 0 728 481 and EP-A 0 509 398 describe quinoxalinone derivatives for the treatment of HIV infections, DE 4341663 describes quinoxalinone derivatives as endothelin receptor antagonists, WO 98/09987 describes quinoxalinone derivatives as thrombin inhibitors, WO 94/11355 describes 3,4-dihydro-1-phenyl-2(1H)-quinoxalinone derivatives for the treatment of cardiovascular disorders and U.S. Pat. No. 3,654,275 describes quinoxaline carboxamides as compounds having anti-inflammatory activity.

It is an object of the present invention to provide medicaments for the treatment of disorders, especially cardiovascular disorders.

The present invention relates to compounds of the formula 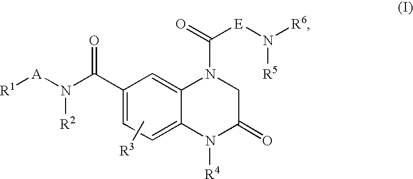
in which
A is a C₁-C₆-alkanediyl chain which is optionally substituted by one or two hydroxy groups,
E is a C₁-C₆-alkanediyl chain,
R¹ is heteroaryl, where heteroaryl is optionally substituted by 1 to 3 substituents independently of one another selected from the group consisting of halogen, hydroxy, amino, trifluoromethyl, nitro, cyano, alkyl, alkoxy, alkylamino, alkoxycarbonyl, aminocarbonyl and alkylaminocarbonyl,
R² is hydrogen, alkyl or cycloalkyl,
R³ is hydrogen, halogen, alkyl or alkoxy,
R⁴ is alkyl or cycloalkyl, where alkyl and cycloalkyl are optionally substituted by 1 to 3 substituents independently of one another selected from the group consisting of halogen, hydroxy, amino, alkyl, alkoxy, alkylamino, alkoxycarbonyl, aminocarbonyl and alkylaminocarbonyl,
R⁵ is hydrogen, alkyl or cycloalkyl,
   and
R⁶ is alkyl, cycloalkyl, heterocyclyl, aryl or heteroaryl, where alkyl, cycloalkyl, heterocyclyl, aryl or heteroaryl are optionally substituted by 1 to 3 substituents independently of one another selected from the group consisting of halogen, hydroxy, amino, trifluoromethyl, nitro, cyano, alkyl which may in turn be substituted by one or two hydroxy groups, or alkoxy, alkylamino, alkoxycarbonyl, aminocarbonyl and alkylaminocarbonyl,
   or
R⁵and R⁶ form together with the nitrogen atom to which they are bonded a nitrogen-containing heterocyclyl ring, where heterocyclyl is optionally substituted by 1 to 3 substituents independently of one another selected from the group consisting of halogen, hydroxy, amino, trifluoromethyl, nitro, cyano, alkyl, alkoxy, alkylamino, alkoxycarbonyl, aminocarbonyl and alkylaminocarbonyl.

The compounds of the invention may also exist in the form of their salts, solvates or solvates of these salts.

The compounds of the invention may exist, depending on their structure, in stereoisomeric forms (enantiomers, diastereomers). The invention therefore relates to the enantiomers or diastereomers and respective mixtures thereof. The stereoisomerically pure constituents can be isolated in a known manner from such mixtures of enantiomers and/or diastereomers.

The invention also relates, depending on the structure of the compounds, to tautomers of the compounds.

Salts which are preferred for the purposes of the invention are physiologically acceptable salts of the compounds of the invention.

Physiologically acceptable salts of the compounds (I) of the invention comprise acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, propionic acid, lactic acid, tartaric acid, malic acid, citric acid, fumaric acid, maleic acid and benzoic acid.

Physiologically acceptable salts of the compounds (I) of the invention also comprise salts of conventional bases such as, for example and advantageously, alkali metal salts (for example sodium and potassium salts), alkaline earth metal salts (for example calcium and magnesium salts) and ammonium salts derived from ammonia or organic amines with 1 to 16 C atoms, such as, for example and preferably, ethylamine, diethylamine, triethylamine, ethyldiisopropylamme, monoethanolamine, diethanolamine, triethanolamine, dicyclohexylamine, dimeihylaminoethanol, procaine, dibenzylamine, N-methylmorpholine, dihydroabiethylamine, arginine, lysine, ethylenediamine and methylpiperidine.

Solvates refer for the purposes of the invention to those forms of the compounds of the invention which form a complex in the solid or liquid state through coordination with solvent molecules. Hydrates are a specific type of solvates in which the coordination takes place with water.

For the purposes of the present invention, unless otherwise specified, the substituents have the following meaning:

Alkyl per se and ‘alk’ and ‘alkyl’ in alkoxy, alkylamino, alkylaminocarbonyl and alkoxycarbonyl stand for a linear or branched alkyl radical with, usually, 1 to 6, preferably 1 to 4, particularly preferably 1 to 3, carbon atoms, by way of example and preferably methyl, ethyl, n-propyl, isopropyl, tert-butyl, n-pentyl and n-hexyl.

Alkoxy is by way of example and preferably methoxy, ethoxy, n-propoxy, isopropoxy, tert-butoxy, n-pentoxy and n-hexoxy.

Alkylamino is an alkylamino radical with one or two alkyl substituents (chosen independently of one another), by way of example and preferably methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

Alkylaminocarbonyl is an alkylaminocarbonyl radical with one or two alkyl substituents (chosen independently of one another), by way of example and preferably methylaminocarbonyl, ethylaminocarbonyl, n-propylaminocarbonyl, isopropylaminocarbonyl, tert-butylaminocarbonyl, n-pentylaminocarbonyl, n-hexyl-aminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl, N-t-butyl-N-methylaminocarbonyl, N-ethyl-N-n-pentyl-aminocarbonyl and N-n-hexyl-N-methylaminocarbonyl.

Alkoxycarbonyl is by way of example and preferably methoxycarbonyl, ethoxycarbonyl, n-propoxycarbonyl, isopropoxycarbonyl, tert-butoxycarbonyl, n-pentoxycarbonyl and n-hexoxycarbonyl.

Alkanediyl is a straight-chain or branched saturated alkanediyl radical with 1 to 6 carbon atoms. A straight-chain or branched alkanediyl radical with 1 to 4 carbon atoms is preferred. Mention may be made by way of example and preferably of methylene, ethane-1,2-diyl, ethane-1,1-diyl, propane-1,3-diyl, propane-1,2-diyl, propane-2,2-diyl, butane-1,4-diyl, butane-1,3-diyl, butane-2,4-diyl, pentane-1,5-diyl, pentane-2,4-diyl, 2-methylpentane-2,4-diyl.

Cycloalkyl is a cycloalkyl group with, usually, 3 to 8, preferably 3 to 6, carbon atoms, by way of example and preferably cyclopropyl, cyclobutyl, cyclopentyl, cyclohexyl and cycloheptyl.

Aryl is a mono-, bi- or tricyclic aromatic, carbocyclic radical with, usually, 6 to 14 carbon atoms, by way of example and preferably phenyl, naphthyl and phenanthrenyl.

Heteroaryl is an aromatic, mono- or bicyclic radical with, usually, 5 to 10, preferably 5 to 6, ring atoms and up to 5, preferably up to 4, heteroatoms from the series S, O and N, by way of example and preferably thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl.

Heterocyclyl is a mono- or polycyclic, preferably mono- or bicyclic, nonaromatic heterocyclic radical with, usually, 4 to 10, preferably 5 to 8, ring atoms and up to 3, preferably up to 2, heteroatoms and/or heteroatomic groups from the series N, O, S, SO, SO₂. The heterocyclyl radicals may be saturated or partially unsaturated. 5- to 8-membered, monocylic saturated heterocyclyl radicals with up to two heteroatoms from the series O, N and S are preferred, such as, by way of example and preferably, tetrahydrofuran-2-yl, pyrrolidin-2-yl, pyrrolidin-3-yl, pyrrolinyl, piperidinyl, morpholinyl, perhydroazepinyl.

Halogen is fluorine, chlorine, bromine and iodine.

A symbol * at a bond means the point of linkage in the molecule.

If radicals in the compounds of the invention are substituted, the radicals may, unless otherwise specified, be substituted one or more times identically or differently. Substitution with up to three identical or different substituents is preferred. Substitution with one substituent is very particularly preferred.

Preference is given to compounds of the formula (I)

in which

A is a C₁-C₆-alkanediyl chain,
E is a C₁-C₆-alkanediyl chain,
R¹ is heteroaryl, where heteroaryl is optionally substituted by 1 to 3 substituents independently of one another selected from the group consisting of halogen, hydroxy, amino, trifluoromethyl, nitro, cyano, alkyl, alkoxy, alkylamino, alkoxycarbonyl, aminocarbonyl and alkylaminocarbonyl,
R² is hydrogen,
R³ is hydrogen,
R⁴ is alkyl or cycloalkyl, where alkyl and cycloalkyl are optionally substituted by 1 to 3 substituents independently of one another selected from the group consisting of alkyl, alkoxy and alkylamino,
R⁵ is hydrogen,
   and
R⁶ is alkyl, cycloalkyl, heterocyclyl, aryl or heteroaryl, where alkyl, cycloalkyl, heterocyclyl, aryl or heteroaryl are optionally substituted by 1 to 3 substituents independently of one another selected from the group consisting of halogen, hydroxy, amino, trifluoromethyl, nitro, cyano, alkyl, alkoxy, alkylamino, alkoxycarbonyl, aminocarbonyl and alkylaminocarbonyl,

Particularly preferred compounds of the formula (I) are those

in which

A is methylene or ethane-1,1-diyl,
E is methylene,
R¹ is 5- or 6-membered heteroaryl, where heteroaryl is optionally substituted by 1 to 3 substituents independently of one another selected from the group consisting of halogen, hydroxy, amino, alkyl, alkoxy, alkylamino, alkoxycarbonyl, aminocarbonyl and alkylaminocarbonyl,
R² is hydrogen,
R³ is hydrogen,
R⁴ is cycloalkyl, where cycloalkyl is optionally substituted by 1 or 2 alkyl substituents,
R⁵ is hydrogen,
   and
R⁶ is alkyl or cycloalkyl, where cycloalkyl is optionally substituted by 1 to 3 substituents independently of one another selected from the group consisting of alkyl, alkoxy, alkylamino, alkoxycarbonyl, aminocarbonyl and alkylaminocarbonyl,

Very particularly preferred compounds of the formula (I) are those

in which

A is ethane-1,1-diyl,
E is methylene,
R¹ is 6-membered heteroaryl, where heteroaryl is optionally substituted by 1 or 2 substituents independently of one another selected from the group consisting of methyl, ethyl, methoxy and ethoxy,
R² is hydrogen,
R³ is hydrogen,
R⁴ is cyclopropyl,
R⁵ is hydrogen,
   and
R⁶ is cyclohexyl or cyclopentyl, where cyclohexyl or cyclopentyl are optionally substituted by 1 or 2 substituents independently of one another selected from the group consisting of methyl, ethyl, methoxy and ethoxy.

Compounds of the formula (I) which are likewise very particularly preferred are those in which A is ethane-1,1-diyl.

Compounds of the formula (I) which are likewise very particularly preferred are those in which R¹-A-is a radical of the formula

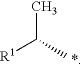

Compounds of the formula (I) which are likewise very particularly preferred are those in which E is methylene.

Compounds of the formula (I) which are likewise very particularly preferred are those in which R¹ is 6-membered heteroaryl, in particular pyridyl.

Compounds of the formula (I) which are likewise very particularly preferred are those in which R² is hydrogen.

Compounds of the formula (I) which are likewise very particularly preferred are those in which R³ is hydrogen.

Compounds of the formula (I) which are likewise very particularly preferred are those in which R⁴ is cycloalkyl, in particular cyclopropyl.

Compounds of the formula (I) which are likewise very particularly preferred are those in which R⁵ is hydrogen.

Compounds of the formula (I) which are likewise very particularly preferred are those in which R⁶ is cyclohexyl or cyclopentyl, where cyclohexyl and cyclopentyl are optionally substituted by 1 or 2 substituents independently of one another selected from the group consisting of methyl and ethyl.

The definitions of radicals indicated specifically in the respective combinations or preferred combinations of radicals are replaced irrespective of the particular combinations indicated for the radicals as desired also by definitions of radicals of another combination.

Combinations of two or more of the abovementioned preferred ranges are very particularly preferred.

The invention further relates to a process for preparing the compounds of the formula (I), characterized in that compounds of the formula 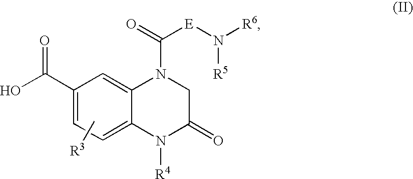
in which
E, R³, R⁴, R⁵ and R⁶ have the meaning indicated above, are reacted
   with compounds of the formula 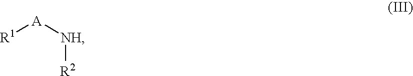
   in which
A, R¹ and R² have the meaning indicated above,
in the presence of conventional condensing agents, where appropriate in the presence of a base.

The reaction takes place where appropriate in inert solvents, preferably in a temperature range from room temperature to 50° C. under atmospheric pressure.

Examples of inert solvents are halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, dioxane, tetrahydrofuran, glycol dimethyl ether or diethylene glycol dimethyl ether, hydrocarbons such as benzene, xylene, toluene, hexane, cyclohexane or petroleum fractions, or other solvents such as nitromethane, ethyl acetate, acetone, dimethylformarnmide, dimethylacetamide, 1,2-dimethoxyethane, dimethyl sulphoxide, acetonitrile or pyridine, with preference for tetrahydrofuran, dimethylformamide or methylene chloride.

Examples of conventional condensing agents are carboduimides such as, for example, N,N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC), N-cyclo-hexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium 3-sulphate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxytri(dimethyl-amino)phosphonium hexafluorophosphate, or O-(benzotriazol-1-yl)-N,N,N′,N′-tetra-methyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), or 1-hydroxy-benzotriazole (HOBt), or benzotriazol-1-yloxytris(dimethylamino)phosphonium hexafluorophosphate (BOP), or mixtures thereof.

Examples of bases are alkali metal carbonates such as, for example, sodium or potassium carbonate, or bicarbonate, or organic bases such as trialkylamines, e.g. triethylamine, N-methylmorpholine, N-methylpiperidine, 4-dimethylaminopyridine or diisopropylethylamine.

The combination of N-(3-dimethylaminoisopropyl)-N′-ethylcarbodiimide hydrochloride (EDC), 1-hydroxybenzotriazole (HOBt) and diisopropylethylamine in methylene chloride is preferred.

Compounds of the formula (III) are known or can be prepared in analogy to known processes.

The compounds of the formula (II) are prepared by reacting compounds of the formula 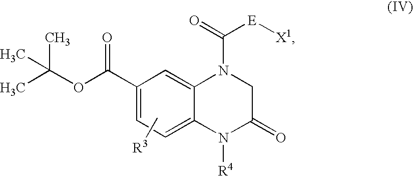
in which
E, R³ and R⁴ have the meaning indicated above, and
X¹ is halogen, preferably bromine or chlorine,
initially with compounds of the formula 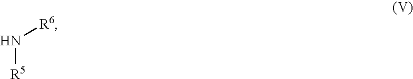
   in which
R⁵ and R⁶ have the meaning indicated above,
and subsequently with trifluoroacetic acid to cleave the tert-butyl ester.

The reaction in the first stage takes place where appropriate in inert solvents, where appropriate in the presence of a base, preferably in a temperature range from 0° C. to 50° C. under atmospheric pressure.

Examples of inert solvents are halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, dioxane, tetrahydrofuran, glycol dimethyl ether or diethylene glycol dimethyl ether, hydrocarbons such as benzene, xylene, toluene, hexane, cyclohexane or petroleum fractions, or other solvents such as nitromethane, ethyl acetate, acetone, dimethylformamide, dimethylacetamide, 1,2-dimethoxyethane, 2-butanone, dimethyl sulphoxide, acetonitrile or pyridine, with preference for tetrahydrofuran or methylene chloride.

Examples of bases are alkali metal hydroxides such as sodium or potassium hydroxide, or alkali metal carbonates such as caesium carbonate, sodium or potassium carbonate, or amides such as lithium diisopropylamide, or other bases such as DBU, triethylamine or diisopropylethylamine, preferably diisopropylethylamine or triethylamine.

The reaction in the second stage takes place in inert solvents, preferably in a temperature range from 0° C. to 50° C. under atmospheric pressure.

Examples of inert solvents are halohydrocarbons such as methylene chloride, trichloromethane, tetrachloromethane, trichloroethane, tetrachloroethane, 1,2-dichloroethane or trichloroethylene, or other solvents dimethylformamide or tetrahydrofuran, with preference for methylene chloride.

Compounds of the formula (V) are known or can be prepared in analogy to known processes.

Compounds of the formula

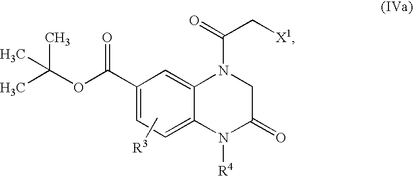

which are compounds of the formula (IV) in which
E is methylene, and
R ³, R⁶ and X¹ have the meaning indicated above,
are prepared by reacting compounds of the formula 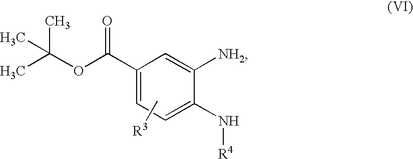
   in which
R³and R⁴ have the meaning indicated above,
with compounds of the formula 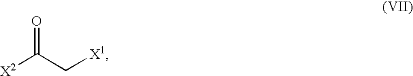
   in which
X¹ has the meaning indicated above, and
X² is halogen, preferably bromine or chlorine.

The reaction takes place in two stages. The reaction in the first stage takes place in inert solvents with 2 equivalents of the compounds of the formula (VII) based on the compounds of the formula (VI), in the presence of 2 equivalents of a base, preferably in a temperature range from 0° C. to 50° C. under atmospheric pressure. The second stage follows without working up the reaction mixture and takes place by adding another base.

Examples of inert solvents are halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, dioxane, tetrahydrofuran, glycol dimethyl ether or diethylene glycol dimethyl ether, hydrocarbons such as benzene, xylene, toluene, hexane, cyclohexane or petroleum fractions, or other solvents such as nitromethane, ethyl acetate, acetone, dimethylformamide, dimethylacetamide, 1,2-dimethoxyethane, 2-butanone, dimethyl sulphoxide, acetonitrile or pyridine, with preference for methylene chloride.

Examples of bases are alkali metal hydroxides such as sodium or potassium hydroxide, or alkali metal carbonates such as caesium carbonate, sodium or potassium carbonate, or amides such as lithium diisopropylamide, or other bases such as DBU, triethylamine or diisopropylethylamine, preferably diisopropylethylamine or triethylamine for the first stage, and preferably DBU for the second stage.

Compounds of the formula (VII) are known or can be prepared in analogy to known processes.

Compounds of the formula (VI) are prepared by reacting compounds of the formula 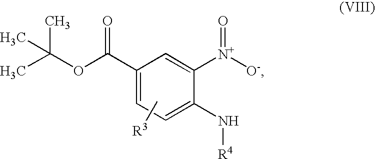
in which
R³ and R⁴ have the meaning indicated above,
with reducing agents in the presence of a catalyst.

The reaction takes place in inert solvents, preferably in a temperature range from room temperature to 50° C. under atmospheric pressure.

Examples of inert solvents are alcohols such as methanol, ethanol, propanol, isopropanol or butanol or ethyl acetate or diethyl ether, with preference for methanol or ethanol.

The reducing agent is, for example, hydrogen; examples of catalysts are tin dichloride, titanium trichloride or palladium on activated carbon. The combination of palladium on activated carbon and hydrogen is preferred.

Compounds of the formula (VIII) are prepared by reacting the compound of the formula 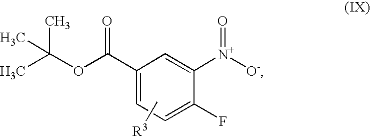
in which
R³ has the meaning indicated above,
with compounds of the formula
   R⁴—NH₂ (X)
   in which
R⁴ has the meaning indicated above,
where appropriate in the presence of a base.

The reaction takes place in inert solvents, preferably in a temperature range from room temperature to 50° C. under atmospheric pressure.

Examples of inert solvents are halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, dioxane, tetrahydrofuran, glycol dimethyl ether or diethylene glycol dimethyl ether, hydrocarbons such as benzene, xylene, toluene, hexane, cyclohexane or petroleum fractions, or other solvents such as nitromethane, ethyl acetate, acetone, dimethylformamide, dimethylacetamide, 1,2-dimethoxyethane, 2-butanone, dimethyl sulphoxide, acetonitrile or pyridine, with preference for tetrahydrofuran or methylene chloride.

Examples of bases are alkali metal hydroxides such as sodium or potassium hydroxide, or alkali metal carbonates such as caesium carbonate, sodium or potassium carbonate, or amides such as lithium diisopropylamide, or other bases such as DBU, triethylamine or diisopropylethylamine, preferably diisopropylethylamine or triethylamine.

Compounds of the formula (IX) and (X) are known or can be prepared in analogy to known processes.

In an alternative method for preparing the compounds of the formula (IV), compounds of the formula 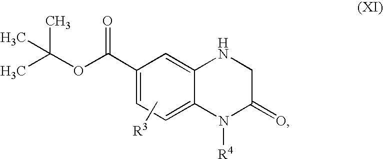
in which
R³ and R⁴ have the meaning indicated above, are reacted with compounds of the formula 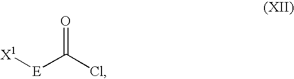
   in which
X¹ and E have the meaning indicated above,
where appropriate in the presence of a base.

The reaction takes place in inert solvents, preferably in a temperature range from room temperature to 50° C. under atmospheric pressure.

Examples of inert solvents are halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, dioxane, tetrahydrofuran, glycol dimethyl ether or diethylene glycol dimethyl ether, hydrocarbons such as benzene, xylene, toluene, hexane, cyclohexane or petroleum fractions, or other solvents such as nitromethane, ethyl acetate, acetone, dimethylformamide, dimethylacetamide, 1,2-dimethoxyethane, 2-butanone, dimethyl sulphoxide, acetonitrile or pyridine, with preference for tetrahydrofuran or methylene chloride.

Examples of bases are alkali metal hydroxides such as sodium or potassium hydroxide, or alkali metal carbonates such as caesium carbonate, sodium or potassium carbonate, or amides such as lithium diisopropylamide, or other bases such as DBU, triethylamine or diisopropylethylamine, preferably triethylamine or diisopropylethylamine.

Compounds of the formula (XI) are known or can be prepared in analogy to known processes (for example as disclosed in EP-A 0 509 398).

Compounds of the formula (XII) are known or can be prepared in analogy to known processes.

The preparation of the compounds of the invention can be illustrated by the following synthesis scheme. 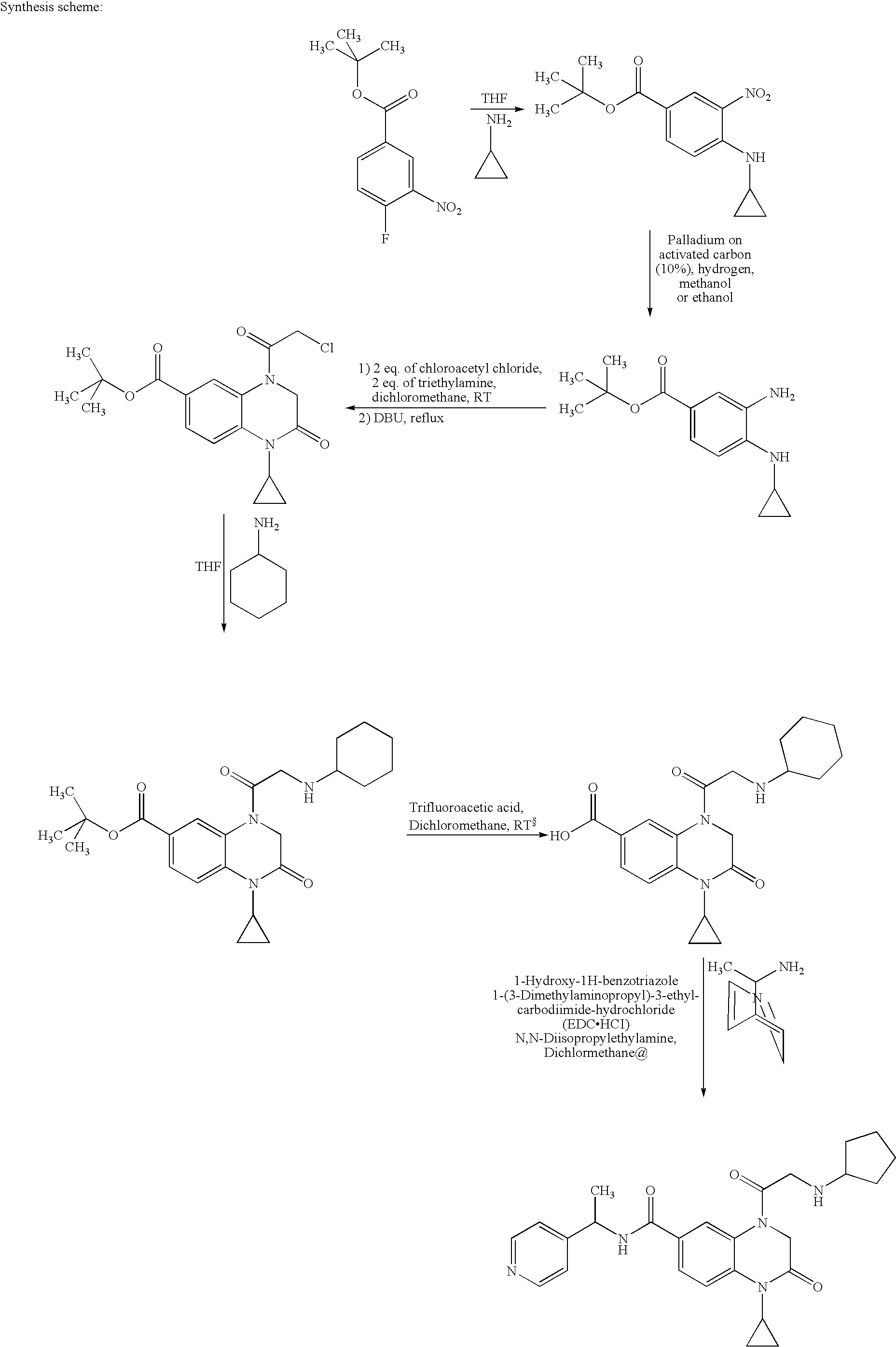
§ F. Zaragoza, H. Stephensen, J. Org. Chem., 1999, 64, 2555-2557 describe the solid-phase synthesis of analogous carboxylic acid derivatives
@ Review of coupling methods: Y. S. Klausner, M. Bodansky, Synthesis 1972, 453-463.

The compounds of the invention show a valuable range of pharmacological and pharmacokinetic effects which could not have been predicted.

They are therefore suitable for use as medicaments for the treatment and/or prophylaxis of diseases in humans and animals.

They are distinguished as agonists of the muscarinic M2 acetycholine receptor.

The compounds of the invention can by reason of their pharmacological properties by employed alone or in combination with other active ingredients for the treatment and/or prophylaxis of cardiovascular diseases, especially of coronary heart disease, angina pectoris, myocardial infarction, stroke, ateriosclerosis, essential, pulmonary and malignant hypertension, heart failure, heart failure, cardiac arrythmias or thromboembolic disorders.

They are additionally suitable for the treatment and/or prophylaxis of disorders of the eye (glaucoma), stomach and intestines (atonias), of the brain (e.g. Parkinson's disease, Alzheimer's disease, chronic sensation of pain), kidney failure or erectile or renal dysfunctions.

The present invention further relates to medicaments which comprise at least one compound of the invention, preferably together with one or more pharmacologically acceptable excipients or carriers, and to the use thereof for the aforementioned purposes.

The active ingredient may have systemic and/or local effects. It can for this purpose be administered in a suitable way such as, for example, by the oral, parenteral, pulmonary, nasal, sublingual, lingual, buccal, rectal, transdermal, conjunctival or otic route or as implant.

The active ingredient can be administered for these routes of administration in suitable administration forms.

Administration forms suitable for oral administration are known ones which deliver the active ingredient rapidly and/or in a modified manner, such as, for example, tablets (uncoated and coated tablets, e.g. with tablets provided with enteric coatings or film-coated tablets), capsules, sugar-coated tablets, granules, pellets, powders, emulsions, suspensions, solutions and aerosols.

Parenteral administration can take place with avoidance of an absorption step (intravenous, intraarterial, intracardiac, intraspinal or intralumbar) or with inclusion of absorption (intramuscular, subcutaneous, intracutaneous, percutaneous, or intraperitoneal). Administration forms suitable for parenteral administration are, inter alia, preparations for injection and infusion in the form of solutions, suspensions, emulsions, lyophilisates and sterile powders.

Examples suitable for the other routes of administration are pharmaceutical forms for inhalation (inter alia powder inhalers, nebulizers), nasal drops/solutions, sprays; tablets or capsules to be administered lingually, sublingually or buccally, suppositories, preparations for the ears and eyes, vaginal capsules, aqueous suspensions (lotions, shaking mixtures), lipophilic suspensions, ointments, creams, milk, pastes, dusting powders or implants, for example stents.

The active ingredients can be converted in a manner known per se into the stated administration forms. This takes place with use of inert, non-toxic, pharmaceutically suitable excipients. These include, inter alia, carriers (for example microcrystalline cellulose), solvents (for example liquid polyethylene glycols), emulsifiers (for example sodium dodecyl sulphate), dispersants (for example polyvinylpyrrolidone), synthetic and natural biopolymers (for example albumin), stabilizers (for example antioxidants such as ascorbic acid), colours (for example inorganic pigments such as iron oxides) or masking flavours and/or odours.

It has generally proved advantageous to administer on parenteral administration amounts of about 0.0001 to 10 mg/kg, preferably about 0.001 to 1 mg/kg, of body weight to achieve effective results. The amount on oral administration is about 0.1 to 10 mg/kg, preferably about 0.5 to 5 mg/kg, of body weight.

It may nevertheless be necessary where appropriate to deviate from the amount mentioned, in particular as a function of the body weight, route of administration, individual response to the active ingredient, nature of the preparation and time or interval level in which administration takes place. Thus, it may be sufficient in some cases to make do with less than the aforementioned minimum amount, whereas in other cases the stated upper limit must be exceeded. It may in the event of administration of larger amounts be advisable to divide these into a plurality of individual doses over the day.

The percentage data in the following tests and examples are, unless otherwise indicated, percentages by weight; parts are parts by weight. Solvent ratios, dilution ratios and concentration data on liquid/liquid solutions are in each case based on volume.

### A. EXAMPLES

Abbreviations:

aq. aqueous
Boc tert-butoxycarbonyl
CDCl₃ deuterochloroform
DMSO dimethyl sulphoxide
DMF dimethylformamide
EDC N′-(3-dimethylaminopropyl)-N-ethylcarbodiimide×HCl
eq. equivalent
ESI electrospray ionization (in MS)
HATU O-(7-azabenzotriazol-1-yl)-N,N,N′,N′-tetramethyluronium hexafluorophosphate
HBTU O-(benzotriazol-1-yl)-N,N,N′,N′-tetramethyluronium hexafluorophosphate
HOBt 1-hydroxy-1H-benzotriazole×H₂O
h hour
HPLC high pressure, high performance liquid chromatography
LC-MS coupled liquid chromatography and mass spectroscopy
MS mass spectroscopy
MeOH methanol
M.p. melting point
NMR nuclear magnetic resonance spectroscopy
Pd/C palladium/carbon
R_{f} retention index (in TLC)
RT room temperature
Rₜ retention time (in HPLC)
sat. saturated
   HPLC and LC-MS Methods:
   Method 1:

Instrument: HP 1100 with DAD detection; column: Kromasil RP-18, 600 mm×2 mm, 3.5 μm; eluent A=5 ml HClO₄/l H₂O, B=ACN; gradient: 0 min 2% B, 0.5 mim 2% B, 4.5 min 90% B, 6.5 min 90% B; flow rate: 0.75 ml/min, temp.: 30 degrees C., WV detection: 210 nm.

Method 2:

Instrument: Micromass Platform LCZ, HP1100; column: Symmetry C18, 50 mm×2.1 mm, 3.5 μm; eluent B: water+0.1% formic acid, eluent A: acetonitrile+0.1% formic acid; gradient: 0.0 min 10% A→4.0 min 90% A→6.0 min 90% A; oven: 40° C., flow rate: 0.5 ml/min, UV detection: 208-400 nm.

Method 3:

Instrument: Finnigan MAT 900S, TSP: P4000,AS3000,UV30000HR; column: Symmetry C 18, 150 mm×2.1 mm, 5.0 μm; eluent C: water, eluent B: water+0.6 g 35% HCl, eluent A: acetonitrile; gradient: 0.0 min 2% A, 49% B, 49% C→2.5 min 95% A, 2.5% B, 2.5% C→5.5 min 2% A, 49% B, 49% C; oven: 70° C., flow rate: 1.2 ml/min, UV detection: 210 nm.

Method 4:

Instrument: Micromass Quattro LCZ, HP1100; column: Symmetry C18, 50 mm×2.1 mm, 3.5 μm; eluent A: acetonitrile+0.1% formic acid, eluent B: water+0.1% formic acid; gradient: 0.0 min 10% A→4.0 min 90% A→6.0 min 90% A; oven: 40° C., flow rate: 0.5 ml/min, UV detection: 208-400 nm.

Method 5:

Prep. HPLC enantiomer separation; Packing material: Daicel Chiralpak AD 250*20 mm ID: AD00CJ-GL001; used in g: 0.13; eluent A: iso-hexane, eluent B: ethanol+0.2% DEA; gradient: 0.0 min 30% A→21 min 30% A; injected volume: 1000 μl temperature: 25° C., flow rate: 10 ml/min, wavelength: 250 nm, range: 1.

Method 6:

Instrument: Micromass Platform LCZ, HP1100; column: Symmetry C18, 50 mm×2.1 mm, 3.5 μm; eluent A: water+0.05% formic acid, eluent B: acetonitrile+0.05% formic acid; gradient: 0.0 min 90% A→4.0min 10% A→6.0 min 10% A; oven: 40° C., flow rate: 0.5 ml/min, UV detection: 208-400 nm.

Method 7:

Instrument: Micromass Quattro LCZ. HP 1100; column: Symmetry C18, 50 mm×2.1 mm, 3.5 μm; eluent A: water+0.05% formic acid, eluent B: acetonitrile+0.05% formic acid; gradient: 0.0 min 90% A→4.0 min 10% A→6.0 min 10% A; oven: 40° C., flow rate 0.5 ml/min, UV detection: 208-400 nm.

Method 8:

Instrument: MS Micromass ZQ; HPLC: Waters Alliance 2790; column: Symmetry C 18, 50 mm×2.1 mm, 3.5 μm; eluent B: acetonitrile+0.05% formic acid, eluent A: water+0.05% formic acid; gradient: 0.0 min 10% B→3.5 min 90% B→5.5 min 90% B; oven: 50° C., flow rate: 0.8 ml/min, UV detection: 210 mn.

Starting Compounds:

### Example I

### tert-Butyl 4-fluoro-3-nitrobenzoate

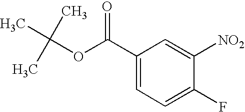

21.5 g (116.15 mmol) of 4-fluoro-3-nitrobenzoic acid and 30.5 g (139.4 mmol) of t-butyl trichloroacetimidate are introduced under an argon atmosphere into 250 ml of diethyl ether. 0.64 g (4.52 mmol) of boron trifluoride-diethyl ether complex is added dropwise, and the mixture is stirred at room temperature for 16 hours.

6 g of solid sodium bicarbonate are added to the reaction mixture, which is concentrated in vacuo. The resulting residue is purified by chromatography on silica gel (mobile phase gradient cyclohexane→cyclohexane/ethyl acetate 1:1).

17.8 g (64% of theory) of product are obtained.

¹H NMR (300 MHz, DMSO-d₆): δ=1.57 (s, 9H), 7.2 (dd, 1H), 8.25-8.3 (m, 1H), 8.52 (dd, 1H)

MS (ESIpos): m/z=242 (M+H)⁺

HPLC (method 1): Rₜ=5.07 min

### Example II

### tert-Butyl 4-(cyclopropylamine)-3-nitrobenzoate

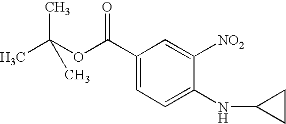

7.8 g (32.3 mmol) of the compound from Example I are introduced into 150 ml of tetrahydrofuran. At 0° C., a solution of 3.88 g (67.9 mmol) of cyclopropylamine in 50 ml of tetrahydrofuran is added. The mixture is stirred at 0° C. for 30 minutes and then at room temperature for 16 hours.

The reaction mixture is concentrated in vacuo. The residue is taken up in 500 ml of ethyl acetate and washed three times with 100 ml of water and once with 100 ml of saturated sodium chloride solution. The solution is dried over sodium sulphate and concentrated in vacuo.

8.95 g (99% of theory) of product are obtained.

¹H NMR (300 MHz, DMSO-d₆): δ=0.64-0.69 (m, 2H), 0.87-0.93 (m, 2H), 1.54 (s, 9H), 2.67-2.71 (m, 1H), 7.44 (d, 1H), 8.01 (dd, 1H), 8.33 (br. s, 1H), 8.54 (d, 1H).

MS (DCI): m/z=296 (N+NH₄)⁺

HPLC (method 1): Rₜ=5.47 min

Alternative synthesis to Example II:

1 g (3.88 mmol) of the compound from Example XXXIII is introduced into tetrahydrofuran (15 ml). 0.44 g (7.76 mmol) of cyclopropylamine is added at room temperature. The solution is stirred at 55° C. for 2 hours. The mixture is then poured into ice-water (50 ml). The precipitated solid is filtered off with suction and dried. 0.65 g (58% of theory) of product is obtained.

The compounds listed in Table 1 are prepared in analogy to the compound from Example II.

TABLE 1

| Example | Structure | Analytical data |
|---|---|---|
| III | 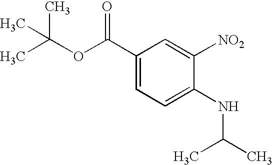 | ¹H-NMR(300 MHz, DMSO-d₆): δ=1.28(d, 6H), 1.53(s, 9H), 3.94-4.01(m, 1H), 7.16(d, 1H), 7.93(dd, 1H),8.15(d, 1H), 8.56(d, 1H)MS(DCI): m/z = 298(M+NH₄)⁺HPLC(Method 1): Rₜ = 5.59 min |
| IV | 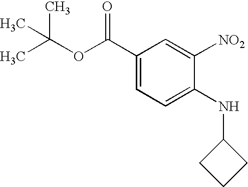 | ¹H-NMR(300 MHz, DMSO-d₆): δ=1.53(s, 9H), 1.73-1.87(m, 2H), 1.98-2.12(m, 2H), 2.4-2.51(m, 2H), 4.13-4.25(m, 1H), 6.98(d, 1H), 7.92(dd,1H), 8.30(d, 1H), 8.55(d, 1H)MS(ESIpos): m/z = 293(M + H)⁺HPLC(Method 1): Rₜ = 5.48 min |
| V | 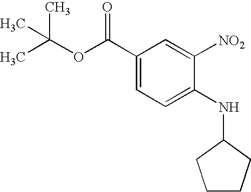 | ¹H-NMR(300 MHz, DMSO-d₆): δ=1.53(s, 9H, 1.58-1.72(m, 6H), 2.08-2.12(m, 2H, 4.07-4.12(m, 1H), 7.16(d, 1H), 7.94(dd, 1H), 8.24(d, 1H),8.55(d, 1H)MS(ESlpos): m/z 307(M + H)⁺HPLC(Method 1): Rₜ = 5.65 min |

### Example VI

### tert-Butyl 3-amino-4-(cyclopropylamine)benzoate

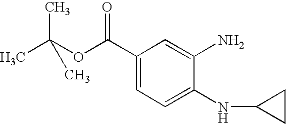

8.85 g (31.8 mmol) of the compound from Example II are introduced into 400 ml of methanol under an argon atmosphere, and 0.30 g (1.33 mmol) of palladium on activated carbon (10% Pd) is added. The mixture is stirred under a hydrogen atmosphere at atmospheric pressure overnight. The mixture is filtered through Celite, and the filtrate is concentrated in vacuo. The product resulting after drying under high vacuum for 2 hours (8.20 g, 94% of theory) is reacted further without delay.

¹H NMR (200 MHz, DMSO-d₆): δ=0.37-0.47 (m, 2H), 0.68-0.80 (m, 2H), 1.48 (s, 9H), 2.35-2.45 (m, 1H), 4.67 (br. s, 2H), 5.64 (br. s, 1H), 6.75 (d, 1H), 7.08 (d, 1H), 7.15 (dd, 1H)

MS (ESIpos): m/z=249 (M+H)⁺

HPLC (method 1): Rₜ=4.18 min

The compounds listed in Table 2 are prepared in analogy to the compound from Example IV. Ethanol is used as solvent. The resulting products are reacted further without delay.

TABLE 2

| Example | Structure | Analytical data |
|---|---|---|
| VII | 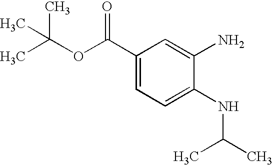 | ¹H-NMR(300 MHz, DMSO-d₆): δ=1.17(d, 6H), 1.48(s, 9H), 3.58-3.69(m, 1H), 4.67(s, 2H), 4.80(d, 1H),6.42(d, 1H), 7.09(d, 1H), 7.13(dd,MS(ESIpos): m/z =251(M + H)⁺HPLC(Method 1): Rₜ = 4.02 min |
| VIII | 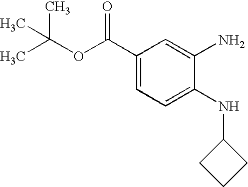 | ¹H-NMR(300 MHz, DMSO-d₆): δ=1.48(s, 9H), 1.68-1.95(m, 4H),2.30-2.42(m, 2H), 3.82-3.94(m,1H), 4.70(s, 2H), 5.30(d, 1H), 6.29(d, 1H), 7.08-7.12(m, 2H)MS(ESIpos): m/z =263(M + H)⁺HPLC(Method 1): Rₜ = 4.08 min |
| IX | 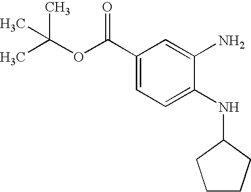 | MS(ESIpos): m/z 277(M + H)⁺HPLC(Method 1): Rₜ = 4.26 min |

### Example X

### tert-Butyl 4-(chloroacetyl)-1-cyclopropyl-2-oxo-1,2,3,4-tetrahydro-6-quinoxaline-carboxylate

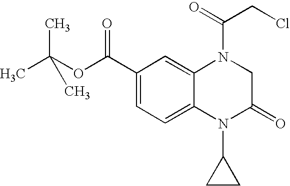

7.90 g (31.8 mmol) of the compound from Example VI are introduced into 200 ml of dichloromethane and, at 0° C., 8.98 g (79.5 mmol) of chloroacetyl chloride are added. The mixture is stirred at room temperature for 30 minutes. At 0° C., 11.2 ml (79.5 mmol) of triethylamine are added. The mixture is stirred at room temperature for 4 hours. Then 7.12 ml (7.26 g, 47.7 mmol) of 1,8-diazabicyclo(5.4.0)undec-7-ene are added, and the mixture is heated to reflux. After 16 hours, the reaction mixture is cooled and concentrated in vacuo.

The residue is purified by chromatography on silica gel (mobile phase gradient cyclohexane→cyclohexane/ethyl acetate 1:1).

4.69 g (62% of theory) of product are obtained as an amorphous solid.

¹H NMR (400 MHz, CDCl₃): δ=0.68-0.72 (m, 2H), 1.16-1.21 (m, 2H), 1.60 (s, 9H), 2.78-2.82 (m, 1H), 4.21 (s, 2H), 4.51 (br. s, 2H), 7.46 (d, 1H), 7.98 (m, 2H)

MS (DCI): m/z=382 (M+NH₄)⁺

HPLC (method 1): Rₜ=4.72 min

### Example XI

### tert-Butyl 4-(chloroacetyl)-1-isopropyl-2-oxo-1,2,3,4-tetrahydro-6-quinoxaline-carboxylate

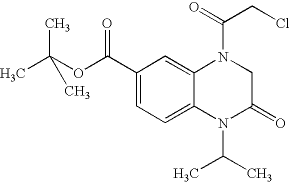

Preparation takes place in analogy to Example X from the appropriate precursors.

The mixture is worked up by washing three times with 100 ml of water and once with 100 ml of saturated sodium chloride solution. The organic phase is dried over sodium sulphate and concentrated in vacuo.

The residue is purified by chromatography on silica gel (mobile phase gradient cyclohexane→cyclohexane/ethyl acetate 2:1).

¹H NMR (300 MHz, DMSO-d₆): δ=1.45 (d, 6H), 1.54 (s, 9H), 4.37 (s, 2H), 4.51 (br. s, 2H), 4.61 (quintet, 1H), 7.49 (d, 1H), 7.81 (dd, 1H), 8.12 (d, 1H)

MS (DCI): m/z=384 (M+NH₄)⁺

HPLC (method 1): Rₜ=4.71 min

### Example XII

### tert-Butyl 4-(chloroacetyl)-1-cyclopentyl-2-oxo-1,2,3,4-tetrahydro-6-quinoxaline-carboxylate

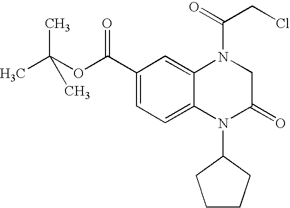

Preparation takes place in analogy to Example X from the appropriate precursors.

The mixture is worked up by washing three times with 100 ml of water and once with 100 ml of saturated sodium chloride solution. The organic phase is dried over sodium sulphate and concentrated in vacuo.

5.63 g (61% of theory) of a dark brown foam are obtained. The product is reacted further without further purification.

DC: R_{f}=0.24 (cyclohexane/ethyl acetate 5:1)

### Example XIII

### tert-Butyl 4-(chloroacetyl)-1-cyclobutyl-2-oxo-1,2,3,4-tetrahydro-6-quinoxalinecarboxylate

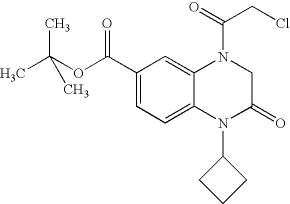

Preparation takes place in analogy to Example X from the appropriate precursors.

The mixture is worked up by washing three times with 100 ml of water and once with 100 ml of saturated sodium chloride solution. The organic phase is dried over sodium sulphate and concentrated in vacuo.

5.42 g (82% of theory) of dark brown foam are obtained. The product is reacted further without further purification.

R_{f}=0.53 (cyclohexane/ethyl acetate 2:1)

### Example XXV

### tert-Butyl 4-(N-cyclopentylglycyl)-1-cyclopropyl-2-oxo-1,2,3,4-tetrahydro-6-quinoxalinecarboxylate

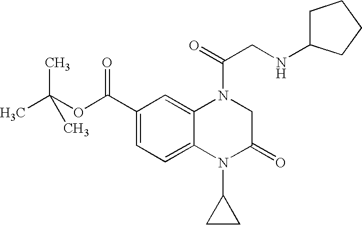

0.10 g (0.27 mmol) of the compound from Example X and 0.07 g (0.82 mmol) of cyclopentylamine are dissolved in 10 ml of tetrahydrofuran, and the mixture is stirred at 40° C. for 6 hours. It is left to stand at room temperature for 12 hours.

The reaction mixture is concentrated in vacuo. The residue is taken up in 20 ml of ethyl acetate and washed twice with 10 ml of saturated sodium chloride solution. The organic phase is dried over sodium sulphate and concentrated in vacuo. The resulting product is reacted further immediately without further purification.

LCMS (method 2): Rₜ=2.85 min

MS (ESIpos): m/z=414 (M+H)⁺

The compounds listed in Table 3 are prepared in analogy to the compound from Example XIV from the appropriate precursors and reacted further without purification.

TABLE 3

| Example | Structure | Analytical data |
|---|---|---|
| XV | 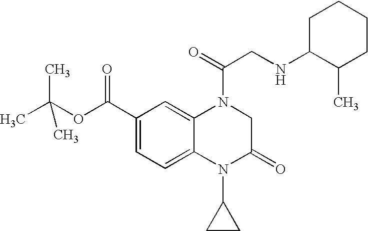 | LCMS(Method 6): Rₜ = 3.29 min MS(ESIpos): m/z = 442 (M + H)⁺ |
| XVI | 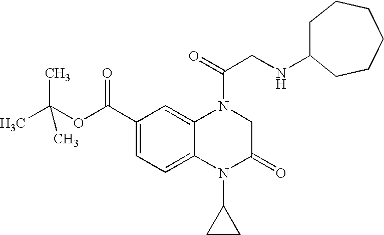 | LCMS(Method 2): Rₜ = 3.06 min MS(ESIpos): m/z = 442 (M + H)⁺ |
| XVII | 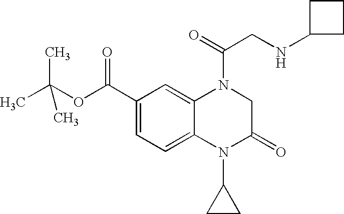 | LCMS(Method 7): Rₜ = 2.9 min MS(ESIpos): m/z = 400 (M + H)⁺ |
| XVIII | 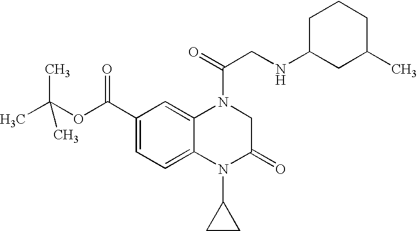 | LCMS(Method 7): Rₜ = 3.32 min MS(ESIpos): m/z = 442 (M + H)⁺ |

### Example XIX

### tert-Butyl 4-(N-cyclohexylglycyl)-1-cyclopropyl-2-oxo-1,2,3,4-tetrahydro-6-quinoxalinecarboxylate

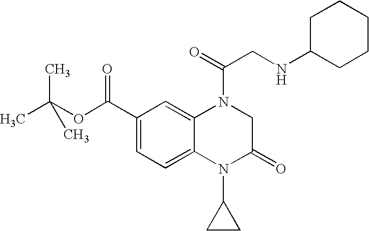

4.5 g (12.33 mmol) of the compound from Example X and 4.24 ml (3.67 g, 37.0 mmol) of cyclohexylamine are dissolved in 175 ml of tetrahydrofuran, and the mixture is left to stand at room temperature for 72 h.

The reaction mixture is concentrated in vacuo. The residue is taken up in 400 ml of ethyl acetate and washed with water (3×100 ml) and once with saturated sodium chloride solution (100 ml). The solution is dried over sodium sulphate and concentrated in vacuo.

It is purified by chromatography on silica gel [mobile phase gradient dichloromethane→dichloromethane/methanol 7.5% (v/v)].

5.30 g (92% of theory) of product are obtained.

¹H NMR (300 MHz, DMSO-d₆): δ=0.50-0.57 (m, 2H), 0.85-1.90 (m, 12H, 1.54 (s, 9H), 2.22-2.37 (m, 1H), 2.78-2.85 (m, 1H), 3.49 (br. s, 2H), 4.42 (s, 2H), 7.52 (d, 1H), 7.83 (dd, 1H), 8.06 (s, 1H)

MS (ESIpos): m/z=428 (M+H)⁺

HPLC (method 1): Rₜ=4.42 min

### Example XX

### tert-Butyl 4-(N-cyclohexylglycyl)-1-isopropyl-2-oxo-1,2,3,4-tetrahydro-6-quinoxalinecarboxylate

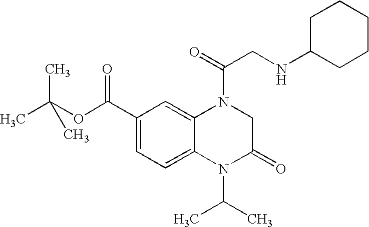

300 mg (0.82 mmol) of the compound from Example XI and 243 mg (2.45 mmol) of cyclohexylamine are stirred in 10 ml of tetrahydrofuran at room temperature for 16 hours.

The reaction mixture is worked up by concentration in vacuo. The residue is purified by chromatography on silica gel [mobile phase gradient: dichloromethane→dichloromethane/methanol 7.5% (v/v)].

LCMS (method 2): Rₜ=3.27 min

MS (ESIpos): m/z=430 (M+H)⁺

### Example XXI

### tert-Butyl 4-(N-cyclopentylglycyl)-1-isopropyl-2-oxo-1,2,3,4-tetrahydro-6-quinoxalinecarboxylate

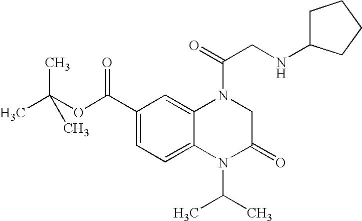

The compound is prepared in analogy to Example XX from the appropriate precursors.

LCMS (method 7): Rₜ=3.16 min

MS (ESIpos): m/z=416 (M+H)⁺

### Example XXII

### tert-Butyl 4-(N-cyclohexylglycyl)-1-cyclopentyl-2-oxo-1,2,3,4-tetrahydro-6-quinoxalinecarboxylate

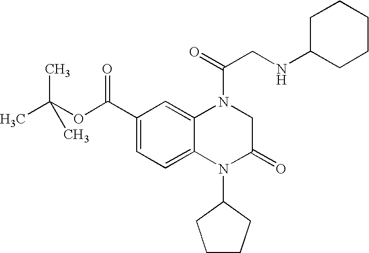

The compound is prepared as described from Example XIX from the appropriate precursors. The resulting crude product is purified by chromatography on silica gel [mobile phase dichloromethane→dichloromethane/methanol 5% (v/v)]. A pale brown oil (410 mg, 21% of theory) is obtained as product.

¹H NMR (400 MHz, DMSO-d₆): δ=0.90-2.10 (m, 27H), 2.31 (m, 1H), 2.42 (m, 1H), 3.53 (br. s, 2H), 4.39 (br., s, 2H), 4.36 (quintet, 2H), 7.43 (d, 1H), 7.79 (dd, 1H), 8.08 (br., s, 1H)

MS (ESIpos): m/z=456 (M+H)⁺

### Example XXIII

### tert-Butyl 4-(N-cyclohexylglycyl)-1-cyclobutyl-2-oxo-1,2,3,4-tetrahydro-6-quinoxalinecarboxylate

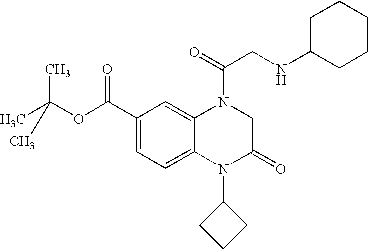

The synthesis takes place as described for the compound in Example XIX.

The resulting crude product is purified by chromatography on silica gel mobile phase gradient [dichloromethane→dichloromethane/methanol 5% (v/v)].

A brown oil (701 mg (21% of theory) is obtained as product.

¹H NMR (400 MHz, DMSO-d₆): δ=0.90-1.24 (m, 6H), 1.54 (s, 9H), 1.45-1.82 (m, 8H), 2.00-2.18 (mn, 2H), 2.22-2.40 (m, 2H), 3.56 (br. s, 2H), 4.37 (br. s, 2H), 4.45 (quintet, 1H), 7.14 (d, 1H), 7.76 (d, 1H), 8.07 (br. s, 1H).

MS (ESIpos): m/z=442 (M+H)⁺

### Example XXIV

### 4-(N-Cyclopentylglycyl)-1-cyclopropyl-2-oxo-1,2,3,4-tetrahydro-6-quinoxalinecarboxylic acid

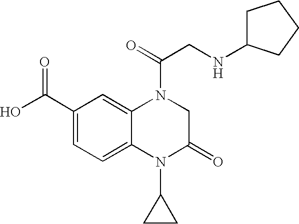

0.11 g (0.27 mmol) of the compound from Example XIX is mixed with 2 ml of a mixture of trifluoroacetic acid and dichloromethane in the ratio 1:1. The mixture is stirred at room temperature for 30 minutes.

The solution is concentrated in vacuo and dried under high vacuum. The residue is taken up in 10 ml of a 1:1 dichloromethane/methanol mixture and stirred with 1 g of solid sodium bicarbonate for 60 minutes. The mixture is diluted with 20 ml of dichloromethane and filtered with suction, and the filtrate is concentrated. The resulting product is reacted further without further purification.

LCMS (method 3): Rₜ=0.84 min

MS (ESIpos): m/z=358 (M+H)⁺

The compounds listed in Table 4 are prepared analogously from the appropriate precursors and either immediately reacted further or purified by preparative HPLC (column material: GROM-SIL 120 OSD4 HE, 10 μm; mobile phase gradient acetonitrile:water 10:90→95:5).

TABLE 4

| Example | Structure | Analytical data |
|---|---|---|
| XXV | 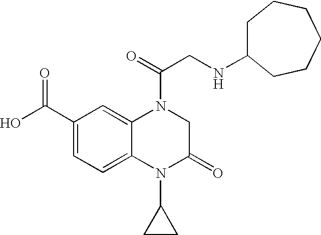 | LCMS(Method 3): Rₜ = 0.83minMS(ESIpos): m/z = 386(M + H)⁺ |
| XXVI | 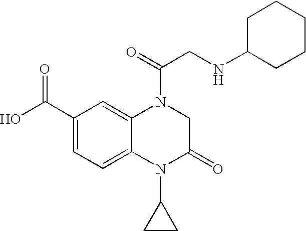 | ¹H-NMR(300 MHz, DMSO-d₆):δ= 0.50-0.63(m, 2H), 0.95-7H), 1.45-1.90(m,5H), 2.40-2.50(m, 1H), 2.78-2.85(m, 1H), 3.67(br. s, 2H),4.43(s, 2H), 7.52(d, 1H), 7.88MS(ESIpos): m/z = 372(M + H)⁺HPLC(Method 1): Rₜ = 3.64min |
| XXVII | 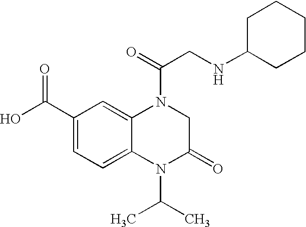 | LCMS(Method 4): Rₜ = 2.64minMS(ESIpos): m/z = 374(M + H)⁺ |
| XXVIII | 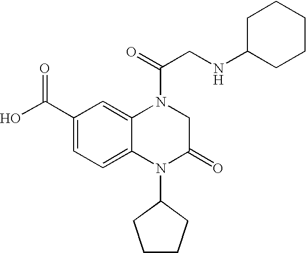 | LCMS(Method 6): Rₜ = 3.17minMS(ESIpos): m/z = 400(M + H)⁺ |
| XXIX | 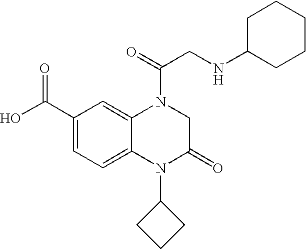 | LCMS(Method 6): Rₜ = 3.03minMS(ESIpos): m/z = 386(M + H)⁺ |
| XXX | 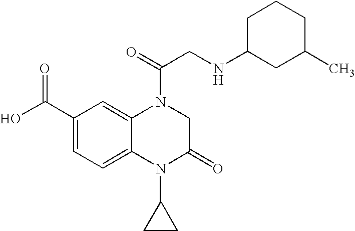 | LCMS(Method 6): Rₜ = 2.75minMS(ESIpos): m/z = 386(M + H)⁺ |
| XXXI | 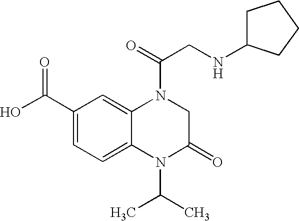 | LCMS(Method 8) Rₜ = 1.50minMS(ESIpos): m/z = = 360(M + H)⁺ |
| XXXII | 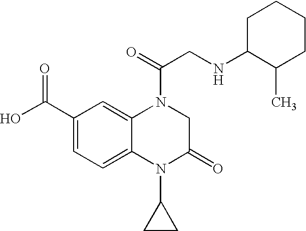 | LCMS(Method 6): Rₜ = 3.48minMS(ESIpos): m/z = 386(M + H)⁺ |

### Example XXXIII

### tert-Butyl 4-chloro-3-nitrobezoate

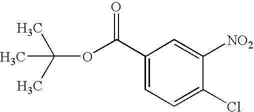

10 g (45.45 mmol) of 4-chloro-3-nitrobenzyl chloride are dissolved in DMF (100 ml). 5.10 g of potassium tert-butoxide are added in portions at room temperature. The solution is stirred at room temperature for one hour. The mixture is then poured in portions into ice-water (500 ml). The precipitated solid is filtered off with suction and dried.

7.1 g (60% of theory) of product are obtained.

¹H NMR (300 MHz, DMSO-d₆): δ=1.59 (s, 9H), 7.9 (dd, 1H), 8.18 (m, 1H), 8.49 (dd, 1H).

MS (ESIpos): m/z=258 (M+H)⁺

HPLC (method 1): Rₜ=5.10 min

| | | Rₜ | Method | Mass | |
|---|---|---|---|---|---|
| Example | Structure | [min] | LCMS | (M + H) | ¹H-NMR |
| XXXIV | 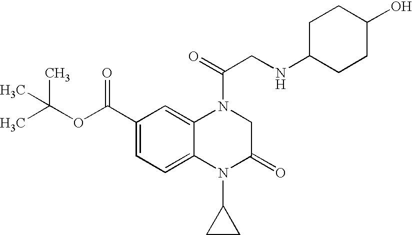 | 2.81 | 8 | 444 | ¹H-NMR(400 MHz, DMSO-d₆): d=0.48-0.60(m, 2H), 0.83-1.22(m, 5H), 1,54(s, 9H), 1.57-1.70(m, 5H), 2.18-2.30(m, 1H), 2.80-2.88(m, 2H), 3.50 (br. s, 2H), 3.55(m, 1H), 4.42(s, 2H), 7.37(d, 1H), 7.52 (dd, 1H), 7.88(dd, 1H). |
| XXXV | 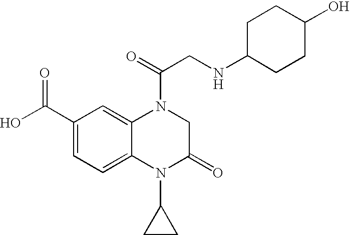 | 1.95 | 8 | 387 | ¹H-NMR(400 MHz, DMSO-d₆): d=0.48-0.60(m, 2H), 0.83-1.22(m, 5H), 1.57-1.70(m, 5H), 2.18-2.30(m, 1H), 2.80-2.88(m, 2H), 3.50(br. s, 2H), 3.55(m, 1H), 4.42 (s, 2H), 7.37(d, 1H), 7.52(dd, 1H), 7.88(dd, 1H), 9.88 (s, 1H). |
| XXXVI | 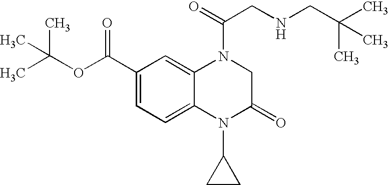 | 2.93 | 1 | 416 | |
| XXXVII | 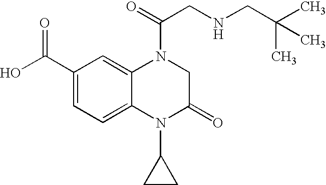 | 1.93 | 1 | 360 | |
| XXXVII | 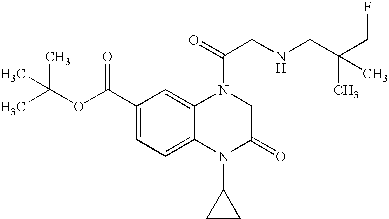 | 3.01 | 8 | 434 | |
| XXXIX | 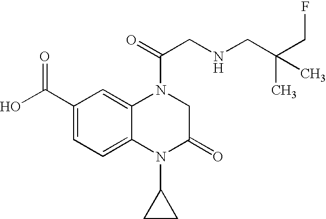 | 1.99 | 1 | 378 | |
| XL | 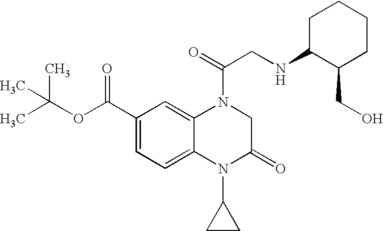 | 3.15 | 1 | 457 | |
| XLI | 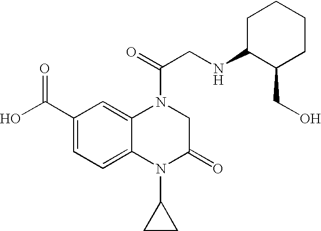 | 2.26 | 1 | 401 | |
| XLII | 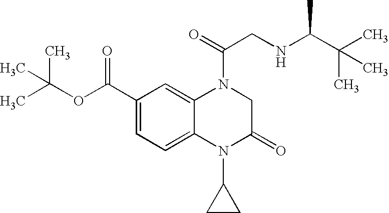 | 2.98 | 1 | 430 | |
| XLIII | 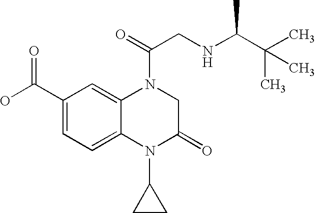 | 1.95 | 1 | 374 | |
| XLIV | 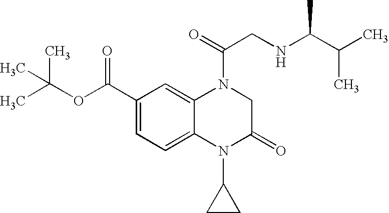 | 2.93 | 1 | 415 | |
| XLV | 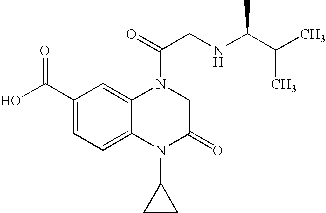 | 1.94 | 1 | 359 | |
| XLVI | 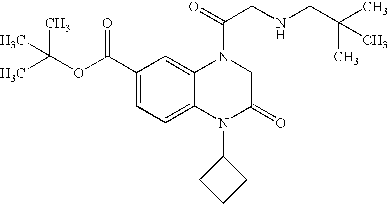 | 2.95 | 8 | 430 | (300 MHz, CDCl₃): d=7.87(d, J=10 Hz, 1H), 7.25(s, 1H), 6.92(d, J =10 Hz, 1H) 4.48-4.39 (m, 3H), 3.64(s, 2H), 2.60(sb, 2H), 2.36(s, 2H), 2.25-2.21(m, 2H), 1.88-1.81(m, 2H), 1.80 (s, 9H), 0.94(s, 9H) |
| XLVII | 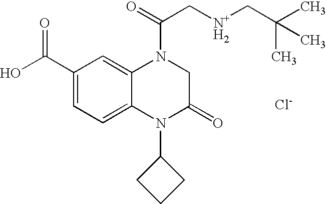 | 2.45 | 8 | 373 (M—HCl) | |
| XLVIII | 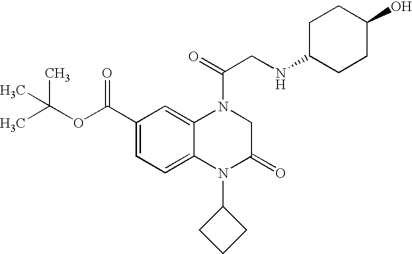 | 2.83 | 8 | 458 | |
| XLIX | 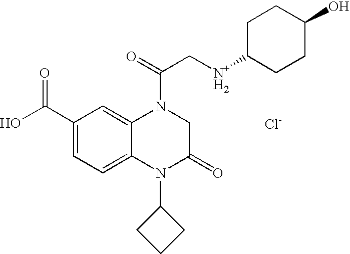 | 1.97 | 8 | 401 (M—HCl) | |
| XLX | 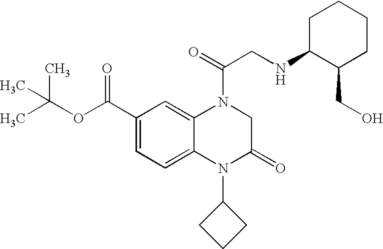 | 2.85 | 8 | 472 | |
| LI | 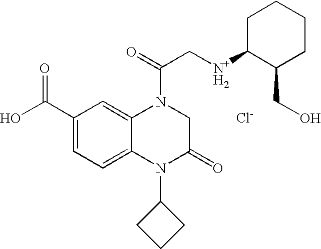 | 2.42 | 8 | 415 (M—HCl) | |

Exemplary Embodiments:

### Example 1

### 4-(N-Cyclohexylglycyl)-1-cyclopropyl-N-(3-furylmethyl)-2-oxo-1,2,3,4-tetrahydro-6-quinoxalinecarboxamide

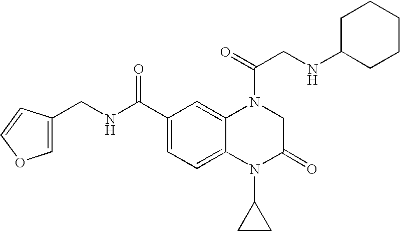

200 mg (0.27 mmol) of the compound of Example XXV are introduced into 10 ml of dichloromethane and, at room temperature, 36.4 mg (0.27 mmol) of 1-hydroxy-1H-benzotriazole and 51.4 mg (0.27 mmol) of 1-(3-dimethylaminopropyl)-3-ethyl-carbodiimide hydrochloride are added. After stirring for 20 minutes, 34.8 mg (0.27 mmol) of N,N-diisopropylethylamine and 26.2 mg (0.27 mmol) of 3-aminomethylfuran are added. The mixture is stirred at room temperature for 16 hours.

The reaction mixture is mixed with 20 ml of dichloromethane and washed once with 20 ml of water and once with 20 ml of saturated sodium chloride solution. It is dried over sodium sulphate and concentrated in vacuo.

The residue is purified by chromatography on silica gel [mobile phase gradient dichloromethane→dichloromethane/methanol 10% (v/v)].

51 mg (42% of theory) of product are obtained.

¹H NMR (300 MHz, DMSO-d₆): δ=0.48-0.57 (m, 2H), 0.79-1.70 (m, 12H), 2.19-2.30 (m, 1H), 2.79-2.87 (m, 1H), 3.51 (s, 2H), 4.31 (d, 2H), 4.41 (s, 2H), 6.45 (s, 1H), 7.50 (d, 1H), 7.58 (s, 2H), 7.83 (d, 1H), 8.02 (s, 1H), 8.80 (t, 1H).

MS (ESIpos): m/z=451 (M+H)⁺

HPLC (method 1): Rₜ=3.92 min

### Example 2

### 4-(N-Cyclobutylglycyl)-1-cyclopropyl-2-oxo-N-[1-(3-pyridinyl)ethyl]-1,2,3,4-tetra-hydro-6-quinoxalinecarboxamide

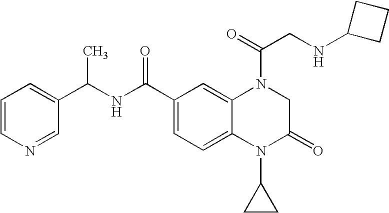

Preparation takes place from the appropriate precursors as described for Example 1.

The dichloromethane/dimethylformamide mixture in the ratio 2:1 is used as solvent.

¹H NMR (300 MHz, DMSO-d₆): δ=0.48-0.57 (m, 2H), 1.02-1.12 (m, 2H), 1.23 (s, 1H), 1.45-1.65 (m, 7H), 1.9-2.0 (m, 2H), 2.79-2.87 (m, 1H), 3.08-3.15 (m, 1H), 3.45 (br. s, 2H), 4.4 (s, 2H), 5.2 (quintet, 1H), 7.35 (dd, 1H), 7.51 (d, 1H), 7.78 (d, 1H), 7.87 (d, 1H), 8.04 (br. s, 1H), 8.44 (dd, 1H), 8.6 (d, 1H), 8.81 (d, 1H).

MS (ESIpos): m/z=448 (M+H)⁺

HPLC (method 1): Rₜ=3.19 min

### Example 3

### 4-(N-Cyclopentylglycyl)-1-cyclopropyl-2-oxo-N-[1-(4-pyridinyl)ethyl]-1,2,3,4-tetrahydro-6-quinoxalinecarboxamide

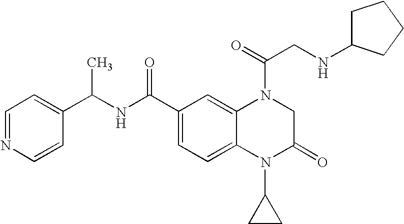

Preparation takes place in analogy to the preparation of the compound from Example 1 from the appropriate precursors.

A dichloromethane/dimethylformamide mixture in the ratio 2:1 is used as solvent.

¹H NMR (300 MHz, DMSO-d₆): δ=0.48-0.58 (m, 2H), 1.05-1.3 (m, 4H), 1.33-1.7 (m, 9H), 2.78-3.0 (m, 2H), 3.5 (s, 2H), 4.42 (s, 2H), 5.14 (quintet, 1H), 7.37 (d, 2H), 7.52 (d, 1H), 7.89 (d, 1H), 8.07 (br. s, 1H), 8.50 (d, 2H), 8.83 (d, 1H).

MS (ESIpos): m/z=462 (M+H)⁺

HPLC (method 1): Rₜ=3.28 min

### Example 4

### 4-(N-Cyclopentylglycyl)-1-cyclopropyl-2-oxo-N-[1-(3-pyridinyl)ethyl]-1,2,3,4-tetrahydro-6-quinoxalinecarboxamide

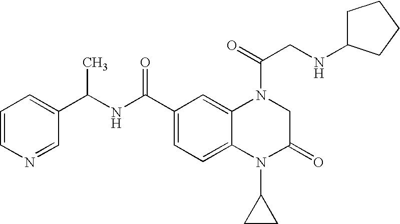

Preparation takes place in analogy to the preparation of the compound from Example 1 from the appropriate precursors.

A dichloromethane/dimethylformamide mixture in the ratio 2:1 is used as solvent.

¹H NMR (300 MHz, DMSO-d₆): δ=0.48-0.58 (m, 2H), 1.05-1.25 (m, 5H), 1.33-1.65 (m, 8H), 2.78-3.0 (m, 2H), 3.48 (br. s, 2H), 4.42 (s, 2H), 5.20 (quintet, 1H), 7.35 (dd, 1H), 7.51 (d, 1H), 7.78 (dt, 1H), 7.87 (d, 1H), 8.05 (br. s, 1H), 8.44 (dd, 1H), 8.60 (d, 1H), 8.82 (d, 1H).

MS (ESIpos): m/z=462 (M+H)⁺

HPLC (method 1): Rₜ=3.28 min

### Example 5

### 4-(N-Cyclohexylglycyl)-1-cyclopropyl-2-oxo-N-[1-(3-pyridinyl)ethyl]-1,2,3,4-tetrahydro-6-quinoxalinecarboxamide

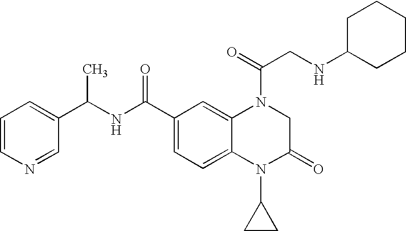

Preparation takes place in analogy to the preparation of the compound from Example 1 from the appropriate precursors.

A dichloromethane/dimethylformamide mixture in the ratio 2:1 is used as solvent.

¹H NMR (200 MHz, DMSO-d₆): δ=0.48-0.60 (m, 2H), 0.8-1.25 (m, 8H), 1.40-1.75 (m, 8H), 2.13-2.36 (m, 1H), 2.78-2.9 (m, 1H), 3.51 (br. s, 2H), 4.43 (s, 2H), 5.13 (quintet, 1H), 7.37 (d, 2H), 7.52 (d, 1H), 7.88 (d, 1H), 8.07 (br. s, 1H), 8.50 (d, 2H), 8.89 (d, 1H).

MS (ESIpos): m/z=476 (M+H)⁺

HPLC (method 1): Rₜ=3.57 min

### Example 6

### 4-(N-Cyclohexylglycyl)-1-cyclopropyl-2-oxo-N-[1-(4-pyridinyl)ethyl]-1,2,3,4-tetrahydro-6-quinoxalinecarboxamide

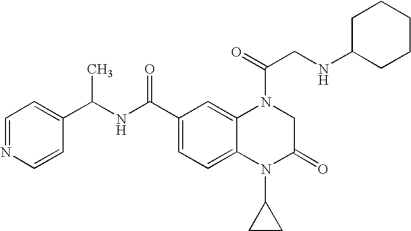

Preparation takes place in analogy to the preparation of the compound from Example 1 from the appropriate precursors.

A dichloromethane/dimethylformamide mixture in the ratio 2:1 is used as solvent.

¹H NMR (400 MHz, DMSO-d₆): δ=0.48-0.60 (m, 2H), 0.83-1.22 (m, 7H), 1.48 (d, 3H), 1.52-1.70 (m, 5H), 2.18-2.30 (m, 1H), 2.80-2.88 (m, 1H), 3.50 (br. s, 2H), 4.42 (s, 2H), 5.13 (quintet, 1H), 7.37 (d, 2H), 7.52 (d, 1H), 7.88 (d, 1H), 8.07 (br. s, 1H), 8.50 (d, 2H), 8.86 (d, 1H).

MS (ESIpos): m/z=476 (M+H)⁺

HPLC (method 1): Rₜ=3.56 min

### Example 7

### (+)-4-(N-Cyclohexylglycyl)-1-cyclopropyl-2-oxo-N-[1-(4-pyridinyl)ethyl]-1,2,3,4-tetrahydro-6-quinoxalinecarboxamide

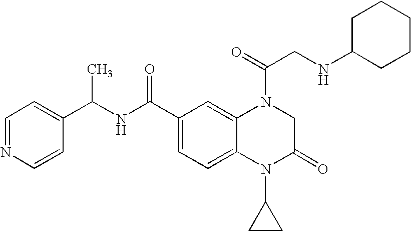

The product is obtained as fraction 2 by chromatographic separation (method 5) of the enantiomers of the compound from Example 6.

¹H NMR (400 MHz, DMSO-d₆): δ=0.52 (s, 2H), 0.80-0.98 (m, 2H), 1.02-1.18 (m, 6H), 1.48 (d, 3H), 1.53-1.70 (m, 4H), 1.78-1.90 (m, 1H), 2.18-2.30 (m, 1H), 2.78-2.88 (m, 1H), 3.51 (br. s, 2H), 4.42 (s, 2H), 5.14 (quintet, 1H), 7.36 (d, 2H), 7.51 (d, 1H), 7.87 (d, 1H), 8.07 (br. s, 1H), 8.50 (d, 2H), 8.86 (d, 1H).

MS (ESIpos): m/z=476 (M+H)⁺

HPLC (method 1): Rₜ=3.41 min

Specific rotation: +29.7° (ethanol, T=20.7° C.)

The second product as fraction 1 is: (−)-4-(N-cyclohexylglycyl)-1-cyclopropyl-2-oxo-N-[1-(4-pyridinyl)ethyl]-1,2,3,4-tetrahydro-6-quinoxalinecarboxamide

¹H NMR (400 MHz, DMSO-d₆): δ=0.52 (s, 2H), 0.80-0.98 (m, 2H), 1.02-1.18 (m, 6H), 1.48 (d, 3H), 1.53-1.70 (m, 4H), 1.78-1.90 (m, 1H), 2.18-2.30 (m, 1H), 2.78-2.88 (m, 1H), 3.51 (br. s, 2H), 4.42 (s, 2H), 5.14 (quintet, 1H), 7.36 (d, 2H), 7.51 (d, 1H), 7.87 (d, 1H), 8.07 (br. s, 1H), 8.50 (d, 2H), 8.86 (d, 1H).

MS (ESIpos): m/z=476 (M+H)⁺

HPLC (method 1): Rₜ=3.41 min

Specific rotation: −36.2° (ethanol, T=20.9° C.)

### Example 8

### 1-Cyclopropyl-4-[N-(2-methylcyclohexyl)glycyl]-2-oxo-N-[1 -(4-pyridinyl)ethyl]-1,2,3,4-tetrahydro-6-quinoxalinecarboxamide

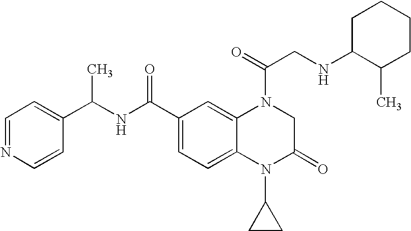

Preparation takes place in analogy to the preparation of the compound from Example 1 from the appropriate precursors.

A dichloromethane/dimethylformamide mixture in the ratio 2:1 is used as solvent.

¹H NMR (300 MHz, DMSO-d₆): δ=0.48-0.60 (m, 2H), 0.76-1.40 (m, 11H), 1.48 (d, 3H), 1.52-1.65 (m, 4H), 1.82-1.95 (m, 1H), 2.80-2.88 (m, 1H), 3.42-3.60 (m, 2H), 4.43 (s, 2H), 5.14 (quintet, 1H), 7.36 (d, 2H), 7.52 (d, 1H), 7.88 (d, 1H), 8.07 (br. s, 1H), 8.49 (d, 2H), 8.83 (d, 1H).

MS (ESIpos): m/z=490 (M+H)⁺

HPLC (method 1): Rₜ=3.67 min

### Example 9

### 1-Cyclopropyl-4-[N-(2-methylcyclohexyl)glycyl]-2-oxo-N-[1-(3-pyridinyl)ethyl]-1,2,3,4-tetrahydro-6-quinoxalinecarboxamide

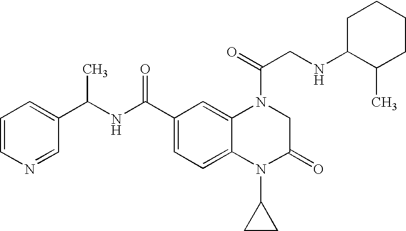

Preparation takes place in analogy to the preparation of the compound from Example 1 from the appropriate precursors.

A dichloromethane/dimethylformamide mixture in the ratio 2:1 is used as solvent.

¹H NMR (300 MHz, DMSO-d₆): δ=0.58-0.68 (m, 2H), 0.72-1.35 (m, 13H), 1.51 (d, 3H), 1.52-1.65 (m, 1H), 1.82-1.95 (m, 1H), 2.76-2.88 (m, 1H), 3.42-3.60 (m, 2H), 4.43 (s, 2H), 5.20 (quintet, 1H), 7.35 (dd, 2H), 7.51 (d, 1H), 7.78 (d, 1H), 7.85 (d, 1H), 8.06 (br. s, 1H), 8.44 (dd, 1H), 8.60 (d, 1H), 8.81 (d, 1H).

MS (ESIpos): m/z=490 (M+H)⁺

HPLC (method 1): Rₜ=3.68 min

### Example 10

### 1-Cyclopropyl-4-[N-(3-methylcyclohexyl)glycyl]-2-oxo-N-[1-(4-pyridinyl)ethyl]-1,2,3,4-tetrahydro-6-quinoxalinecarboxamide

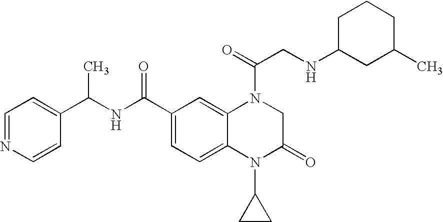

Preparation takes place in analogy to the preparation of the compound from Example 1 from the appropriate precursors.

A dichloromethane/dimethylformamide mixture in the ratio 2:1 is used as solvent.

¹H NMR (300 MHz, DMSO-d₆): δ=0.48-0.58 (m, 2H), 0.68-0.95 (m, 5H), 1.02-1.38 (m, 6H), 1.48 (d, 3H), 1.45-1.72 (m, 4H), 2.20-2.32 (m, 1H), 2.79-2.88 (m, 1H), 3.42-3.58 (m, 2H), 4.42 (s, 2H), 5.14 (quintet, 1H), 7.36 (d, 2H), 7.51 (d, 1H), 7.88 (d, 1H), 8.08 (br. s, 1H), 8.50 (dd, 2H), 8.83 (d, 1H).

MS (ESIpos): m/z=490 (M+H)⁺

HPLC (method 1): Rₜ=3.71 min

### Example 11

### 1-Cyclopropyl-4-[N-(3-methylcyclohexyl)glycyl]-2-oxo-N-[1-(3-pyridinyl)ethyl]-1,2,3,4-tetrahydro-6-quinoxalinecarboxamide

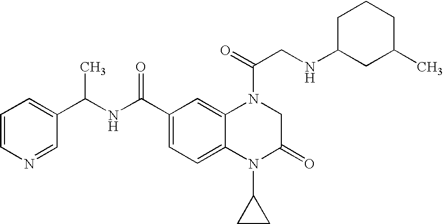

Preparation takes place in analogy to the preparation of the compound from Example 1 from the appropriate precursors.

A dichloromethane/dimethylformamide mixture in the ratio 2:1 is used as solvent.

¹H NMR (300 MHz, DMSO-d₆):δ=0.48-0.58 (m, 2H), 0.68-0.95 (m, 5H), 1.02-1.38 (m, 6H), 1.51 (d, 3H), 1.53-1.75 (m, 4H), 2.20-2.32 (m, 1H), 2.79-2.88 (m, 1H), 3.42-3.58 (m, 2H), 4.41 (s, 2H), 5.20 (quintet, 1H), 7.34 (dd, 1H), 7.51 (d, 1H), 7.78 (d, 1H), 7.86 (d, 1H), 8.06 (br. s, 1H), 8.44 (dd, 1H), 8.60 (d, 1H), 8.81 (d, 1H).

MS (ESIpos): m/z=490 (M+H)⁺

HPLC (method 1): Rₜ=3.72 min

### Example 12

### 4-(N-Cyclohexylglycyl)-1-cyclopropyl-2-oxo-N-(3-thienylmethyl)-1,2,3,4-tetrahydro-6-quinoxalinecarboxamide

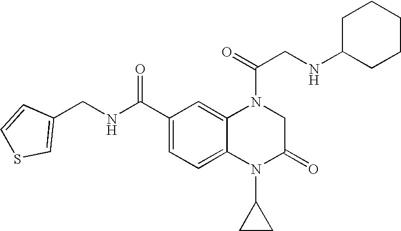

Preparation takes place in analogy to the preparation of the compound from Example 1 from the appropriate precursors.

A dichloromethane/dimethylformamide mixture in the ratio 2:1 is used as solvent.

¹H NMR (300 MHz, DMSO-d₆): δ=0.48-0.58 (m, 2H), 0.82-0.95 (m, 2H), 1.05-1.18 (m, 5H), 1.23 (s, 1H), 1.45-1.72 (m, 5H), 2.20-2.32 (m, 1H), 2.79-2.88 (m, 1H), 3.51 (s, 2H), 4.42 (s, 2H), 4.47 (d, 2H), 7.08 (dd, 1H), 7.29-7.33 (m, 1H), 7.45-7.50 (m, 1H), 7.52 (s, 1H), 7.85 (d, 1H), 8.04 (br. s, 1H), 8.93 (t, 1H).

MS (ESIpos): m/z=467 (M+H)⁺

HPLC (method 1): Rₜ=4.05 min

### Example 13

### 4-(N-Cyclohexylglycyl)-1-isopropyl-2-oxo-N-[1-(4)pyridinyl)ethyl]-1,2,3,4-tetrahydro-6-quinoxalinecarboxamide

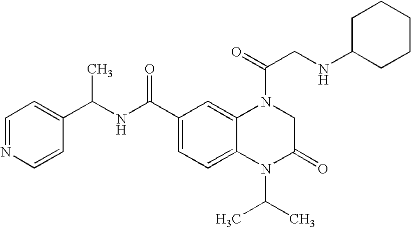

Preparation takes place in analogy to the preparation of the compound from Example 1 from the appropriate precursors.

A dichloromethane/dimethylformamnide mixture in the ratio 5:1 is used as solvent.

¹H NMR (200 MHz, DMSO-d₆): δ=0.70-1.85 (m, 12H), 1.17 (d, 3H), 1.47 (t, 6H), 3.54 (br. s, 2H), 4.37 (s, 2H), 4.60 (quintet, 1H), 5.14 (quintet, 1H), 7.36 (d, 2H), 7.50 (d, 1H), 7.85 (d, 1H), 8.10 (br. s, 1H), 8.50 (d, 2H), 8.92 (d, 1H).

MS (ESIpos): m/z=478 (M+H)⁺

HPLC (method 1): Rₜ=3.63 min

### Example 14

### 4-(N-Cycloheptylglycyl)-1-cyclopropyl-2-oxo-N-[1-(4-pyridinyl)ethyl]-1,2,3,4-tetrahydro-6-quinoxalinecarboxamide

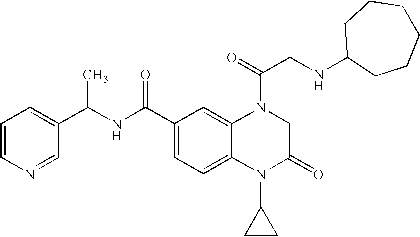

Preparation takes place in analogy to the preparation of the compound from Example 1 from the appropriate precursors.

¹H NMR (400 MHz, DMSO-d₆): δ=0.53 (s, 2H), 1.03-1.65 (m, 16H), 1.48 (d, 3H), 2.79-2.87 (m, 1H), 3.49 (br. s, 2H), 4.42 (s, 2H), 5.14 (quintet, 1H), 7.36 (d, 2H), 7.52 (d, 1H), 7.88 (d, 1H), 8.08 (br. s, 1H), 8.50 (d, 2H), 8.86 (d, 1H).

MS (ESIpos): m/z=490 (M+H)⁺

HPLC (method 1): Rₜ=3.58 min

### Example 15

### 4-(N-Cycloheptylglycyl)-1-cyclopropyl-2-oxo-N-[1-(3-pyridinyl)ethyl]-1,2,3,4-tetrahydro-6-quinoxalinecarboxamide

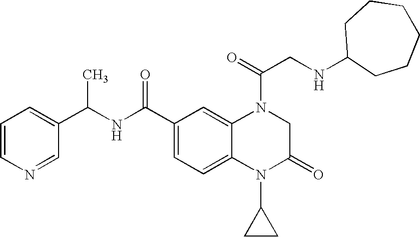

Preparation takes place in analogy to the preparation of the compound from Example 1 from the appropriate precursors.

A dichloromethane/dimethylformamide mixture in the ratio 2:1 is used as solvent.

¹H NMR (400 MHz, DMSO-d₆): δ=0.52 (s, 2H), 1.03-1.88 (m, 16H), 1.51 (d, 3H), 2.80-2.85 (m, 1H), 3.47 (br. s, 2H), 4.41 (s, 2H), 5.19 (quintet, 1H), 7.34 (dd, 1H), 7.65 (dd, 2H), 7.85 (d, 1H), 8.06 (br. s, 1H), 8.63 (dd, 2H), 8.60 (d, 1H).

MS (ESIpos): m/z=490 (M+H)⁺

HPLC (method 1): Rₜ=3.58 min

### Example 16

### 4-(N-Cyclohexylglycyl)-1-cyclopropyl-2-oxo-N-[1-(3-pyridinyl)ethyl]-1,2,3,4-tetrahydro-6-quinoxalinecarboxamide

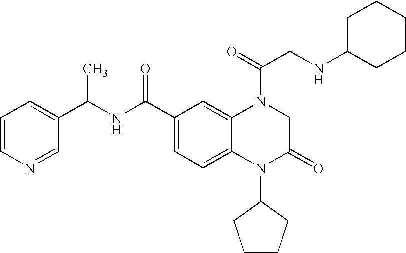

Preparation takes place in analogy to the preparation of the compound from Example 1 from the appropriate precursors.

A dichloromethane/dimethylformamide mixture in the ratio 2:1 is used as solvent.

¹H NMR (400 MHz, DMSO-d₆): δ=0.82-1.20 (m, 6H), 1.51 (d, 3H), 1.53-2.05 (m, 13H), 2.25-2.38 (m, 1H), 3.58 (s, 2H), 4.38 (br. s, 2H), 4.63 (quintet, 1H), 5.20 (quintet, 1H), 7.34 (dd, 1H), 7.60 (dd, 2H), 7.78 (d, 1H), 8.09 (br. s, 1H), 8.44 (d, 1H), 8.60 (d, 1H), 8.86 (d, 1H).

MS (ESIpos): m/z=504 (M+H)⁺

HPLC (method 1): Rₜ=3.79 min

### Example 17

### 1-Cyclobutyl-4-(N-cyclohexylglycyl)-2-oxo-N-[1-(4-pyridinyl)ethyl]-1,2,3,4-tetrahydro-6-quinoxalinecarboxamide

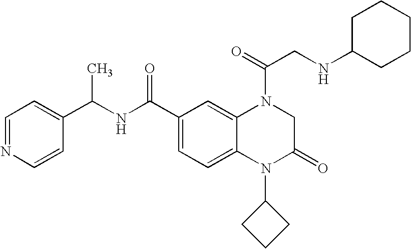

Preparation takes place in analogy to the preparation of the compound from Example 1 from the appropriate precursors.

A dichloromethane/dimethylformamide mixture in the ratio 2:1 is used as solvent.

¹H NMR (400 MHz, DMSO-d₆): δ=0.80-1.13 (m, 8H), 1.48 (d, 3H), 1.49-2.12 (m, 9H), 2.25-2.33 (m, 1H), 3.55 (br. s, 2H), 4.30-4.50 (m, 3H), 5.13 (quintet, 1H), 7.13 (d, 1H), 7.81 (d, 1H), 7.90 (dd, 4H), 8.08 (br. s, 1H), 8.87 (d, 1H).

MS (ESIpos): m/z=490 (M+H)⁺

HPLC (method 1): Rₜ=3.64 min

### Example 18

### 1-Cyclobutyl-4-(N-cyclohexylglycyl)-2-oxo-N-[1-(3-pyridinyl)ethyl]-1,2,3,4-tetrahydro-6-quinoxalinecarboxamide

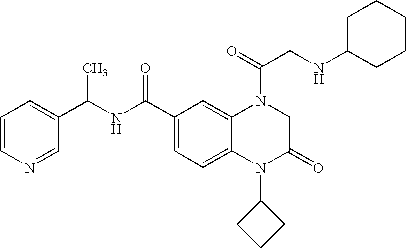

Preparation takes place in analogy to the preparation of the compound from Example 1 from the appropriate precursors.

A dichloromethane/dimethylformamide mixture in the ratio 2:1 is used as solvent.

¹H NMR (400 MHz, DMSO-d₆): δ=0.80-1.30 (m, 6H), 1.51 (d, 3H), 1.52-1.92 (m, 9H), 2.00-2.18 (m, 2H), 2.22-2.36 (m, 1H), 3.55 (br. s, 2H), 4.37 (br. s, 2H), 4.45 (quintet, 1H), 5.19 (quintet, 1H), 7.13 (d, 1H), 7.32-7.37 (m, 1H), 7.77-7.81 (m, 2H), 8.07 (br. s, 1H), 8.84 (d, 1H), 8.60 (d, 1H), 8.85 (d, 1H).

MS (ESIpos): m/z=490 (M+H)⁺

HPLC (method 1): Rₜ=3.63 min

### Example 19

### 4-(N-Cyclohexylglycyl)-1-cyclopropyl-N-[1-(6-methyl-3-pyridinyl)ethyl]-2-oxo-1,2,3,4-tetrahydro-6-quinoxalinecarboxamide

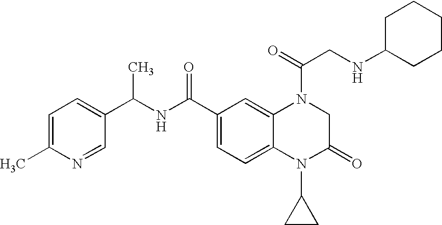

Preparation takes place in analogy to the preparation of the compound from Example 1 from the appropriate precursors.

¹H NMR (400 MHz, DMSO-d₆): δ=0.52 (s, 2H), 0.80-1.20 (m, 8H), 1.49 (d, 3H), 1.52-1.92 (m, 5H), 2.15-2.30 (m, 1H), 2.43 (s, 3H), 2.82 (s, 1H), 3.49 (br. s, 2H), 4.42 (s, 2H), 5.15 (quintet, 1H), 7.19 (d, 1H), 7.50 (d, 1H), 7.66 (d, 1H), 7.85 (d, 1H), 8.04 (br. s, 1H), 8.45 (s, 1H), 8.78 (d, 1H).

MS (ESIpos): m/z=490 (M+H)⁺

HPLC (method 1): Rₜ=3.50 min

### Example 20

### 4-(N-Cyclohexylglycyl)-1-cyclopropyl-N-[1-(6-methoxy-3-pyridinyl)ethyl]-2-oxo-1,2,3,4-tetrahydro-6-quinoxalinecarboxamide

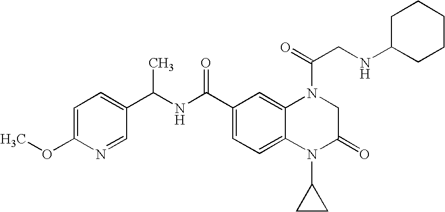

Preparation takes place in analogy to the preparation of the compound from Example 1 from the appropriate precursors.

¹H NMR (400 MHz, DMSO-d₆): δ=0.51 (s, 2H), 0.80-0.98 (m, 2H), 1.00-1.20 (m, 6H), 1.48 (d, 3H), 1.52-1.89 (m, 5H), 2.15-2.30 (m, 1H), 2.82 (s, 1H), 3.49 (br. s, 2H), 3.81 (s, 3H), 4.41 (s, 2H), 5.14 (quintet, 1H), 6.78 (d, 1H), 7.49 (d, 1H), 7.73 (dd, 1H), 7.83 (d, 1H), 8.04 (br. s, 1H), 8.16 (d, 1H), 8.75 (d, 1H).

MS (ESIpos): m/z=506 (M+H)⁺

HPLC (method 1): Rₜ=3.58 min

### Example 21

### 4-(N-Cyclohexylglycyl)-1-cyclopropyl-2-oxo-N-(3-pyridinylmethyl]-1,2,3,4-tetrahydro-6-quinoxalinecarboxamide

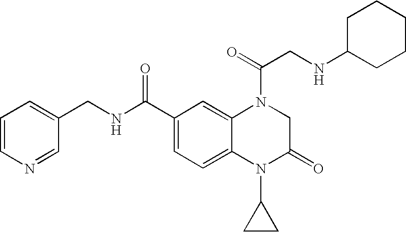

Preparation takes place in analogy to the preparation of the compound from Example 1 from the appropriate precursors.

The crude product is purified by preparative HPLC chromatography [column material: GROM-SIL 120 OSD4 HE, 10 μm, mobile phase gradient acetonitrile/water 10:90→95:5 (v/v)].

¹H NMR (300 MHz, DMSO-d₆): δ=0.53 (m, 2H), 0.79-1.28 (m, 6H), 1.40-1.76 (m, 6H), 2.26 (m, 1H), 2.82 (m, 1H), 3.51 (s, 2H), 4.42 (s, 2H), 4.51 (d, 2H), 7.35 (dd, 1H), 7.52 (d, 1H), 7.72 (dt, 1H), 7.86 (d, 1H), 8.05 (br. s, 1H), 8.45 (dd, 1H), 8.56 (d, 1H), 9.05 (dd, 1H).

MS (ESIpos): m/z=462 (M+H)⁺

### Example 22

### 4-N-[(trans-4-Cyclohexanoyl)glycyl]-1-cyclopropyl-2-oxo-N-[1-S-(4-pyridinyl)-ethyl]-1,2,3,4-tetrahydro-6-quinoxalinecarboxamide

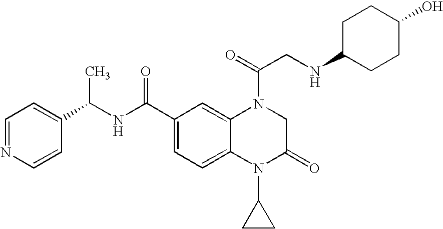

Preparation takes place in analogy to the preparation of the compound from Example 1 from the appropriate precursors. The residue is purified by preparative HPLC (acetonitrile/water).

¹H NMR (400 MHz, DMSO-d₆): δ=0.48-0.60 (m, 2H), 0.83-1.22 (m, 5H), 1.48 (d, 3H), 1.52-1.70 (m, 5H), 2.18-2.30 (m, 1H), 2.80-2.88 (m, 2H), 3.50 (br. s, 2H), 3.55 (m, 1H), 4.42 (s, 2H), 5.13 (quintet, 1H), 7.37 (d, 2H), 7.52 (d, 1H), 7.88 (d, 1H), 8.07 (br. s, 1H), 8.50 (d, 2H), 8.86 (d, 1H).

MS (ESIpos): m/z=492 (M+H)⁺

HPLC (method 1): Rₜ=3.43 min

### Example 23

### 4-N-[(2,2-Dimethylbutyl)glycyl]-1-cyclopropyl-2-oxo-N-[1-S-(4-pyridinyl)ethyl]-1,2,3,4-tetrahydro-6-quinoxalinecarboxamide

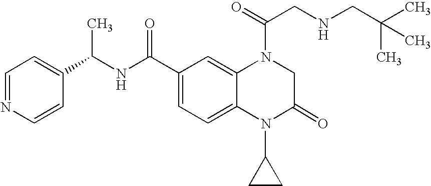

Preparation takes place in analogy to the preparation of the compound from Example 1 from the appropriate precursors. The residue is purified by preparative HPLC (acetonitrile/water).

¹H NMR (400 MHz, DMSO-d₆): δ=0.56 (m, 2H), 0.82 (s, 9H), 1.11 (m, 2H), 1.48 (d, 3H), 2.25 (br. s, 1H), 2.85 (m, 2H), 3.50 (s, 2H), 4.42 (s, 2H), 5.13 (quintet, 1H), 7.37 (d, 2H), 7.51 (d, 1H), 7.88 (d, 1H), 8.07 (br. s, 1H), 8.50 (d, 2H), 8.86 (d, 1H).

MS (ESIpos): m/z=464 (M+H)⁺

HPLC (method 1): Rₜ=3.42 min

### Example 24

### 4-N-[(1-S-Methyl-2,2-dimethylbutyl)glycyl]-1-cyclopropyl-2-oxo-N-[1-S-(4-pyridinyl)ethyl]-1,2,3,4-tetrahydro-6-quinoxalinecarboxamide

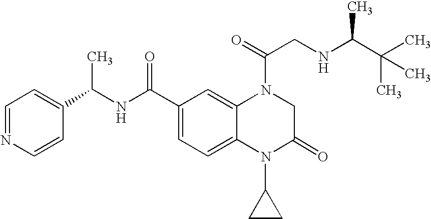

Preparation takes place in analogy to the preparation of the compound from Example 1 from the appropriate precursors. The residue is purified by preparative HPLC (acetonitrile/water).

¹H NMR (400 MHz, DMSO-d₆): δ=0.56 (m, 2H), 0.78 (s, 9H), 1.11 (m, 2H), 1.49 (d, 3H), 2.12 (d, 3H), 2.25 (br. s, 1H), 2.85 (m, 1H), 2.90 (m, 1H), 3.50 (s, 2H), 4.42 (s, 2H), 5.13 (quintet, 1H), 7.37 (d, 2H), 7.51 (d, 1H), 7.88 (d, 1H), 8.07 (br. s, 1H), 8.50 (d, 2H), 8.86 (d, 1H).

MS (ESIpos): m/z=478 (M+H)⁺

HPLC (method 1): Rₜ=3.53 min

### Example 25

### 4-N-[(1-S-Methyl-(2-methylbutyl)glycyl]-1-cyclopropyl-2-oxo-N-[1-S-(4-pyridinyl)-ethyl]-1,2,3,4-tetrahydro-6-quinoxalinecarboxamide

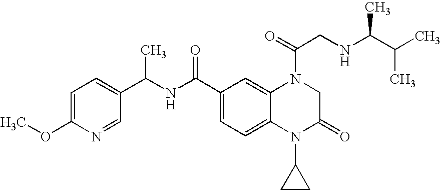

Preparation takes place in analogy to the preparation of the compound from Example 1 from the appropriate precursors. The residue is purified by preparative HPLC (acetonitrile/water).

¹H NMR (400 MHz, DMSO-d₆): δ=0.56 (m, 2H), 0.74 (d, 6H), 1.11 (m, 2H), 1.25 (m, 1H), 1.49 (d, 3H), 2.12 (d, 3H), 2.22 (br. s, 1H), 2.85 (m, 1H), 2.90 (m, 1H), 3.50 (s, 2H), 4.42 (s, 2H), 5.13 (quintet, 1H), 7.37 (d, 2H), 7.51 (d, 1H), 7.88 (d, 1H), 8.07 (br. s, 1H), 8.50 (d, 2H), 8.86 (d, 1H).

MS (ESIpos): m/z=464 (M+H)⁺

HPLC (method 1): Rₜ=3.40 min

### Example 26

### 4-N-[(1-S-Methyl-(2-methylbutyl)glycyl]-1-cyclopropyl-2-oxo-N-[(3-pyridinyl-4-methoxy)ethyl]-1,2,3,4-tetrahydro-6-quinoxalinecarboxamide

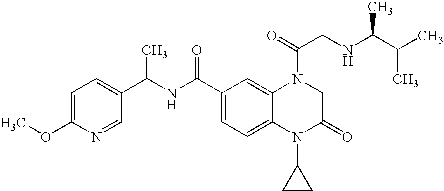

Preparation takes place in analogy to the preparation of the compound from Example 1 from the appropriate precursors. The residue is purified by preparative HPLC (acetonitrile/water).

¹H NMR (400 MHz, DMSO-d₆): δ=0.57 (m, 2H), 0.73 (d, 6H), 1.12 (m, 2H), 1.25 (m, 1H), 1.49 (d, 3H), 2.15 (d, 3H), 2.22 (br. s, 1H), 2.85 (m, 1H), 2.90 (m, 1H), 3.23 (s, 3H), 3.50 (s, 2H), 4.42 (s, 2H), 5.06 (quintet, 1H), 7.49 (d, 2H), 7.52 (m, 1H), 7.83 (d, 1H), 8.20 (d, 1H), 8.85 (d, 1H).

MS (ESIpos): m/z=464 (M+H)⁺

HPLC (method 1): R_{f}=3.40 min

### Example 27

### 4-N-{[2,2-Dimethyl-(3-fluoro)butyl]glycyl}-1-cyclopropyl-2-oxo-N-[1-S-(4-pyridinyl)ethyl]-1,2,3,4-tetrahydro-6-quinoxalinecarboxamide

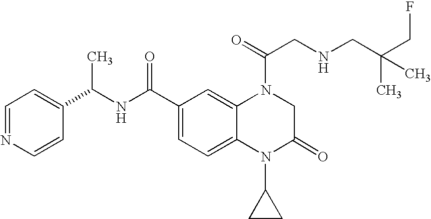

Preparation takes place in analogy to the preparation of the compound from Example 1 from the appropriate precursors. The residue is purified by preparative HPLC (acetonitrile/water).

¹H NMR (400 MHz, DMSO-d₆): δ=0.56 (m, 2H), 1.10 (m, 8H), 1.61 (d, 3H), 2.90 (m, 4H), 4.15 (s, 1H), 4.42 (s, 2H), 5.13 (m, 1H), 7.37 (d, 2H), 7.51 (d, 1H), 7.88 (d, 1H), 8.07 (br. s, 1H), 8.50 (d, 2H), 8.86 (d, 1H).

MS (ESIpos): m/z=482 (M+H)⁺

HPLC (method 1): Rₜ=3.35 min

| | | Rₜ | Method | Mass | |
|---|---|---|---|---|---|
| Example | Structure | [min] | LCMS | (M + H) | ¹H-NMR |
| 28 | 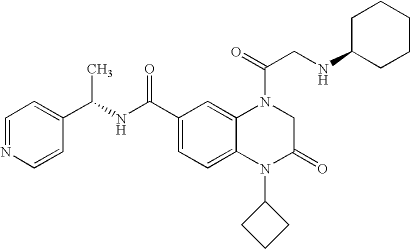 | 3.7 | 1 | 490 | |
| 29 | 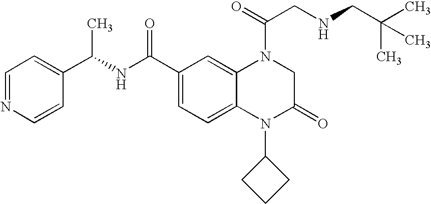 | 3.6 | 1 | 478 | (300 MHz,(CDl3): d=8.58(d, J=5.1 Hz, 2H, 7.96-7.95 (m, 1H, 7.68(d, 7.4 Hz, 1H), 7.28(d, J=5.1 Hz, 2H, 6.95(d, J=8.5 Hz, 1H, 6.41(d, 7.4 Hz, 1H, 5.34-5.24(m, 1H, 4.46-4.37(m, 3H), 3.66(s, 2H, 2.59 (sb, 28), 2.38(s, 2H), 2.29-2.22(m, 28), 1.88-1.81(m, 28), 1.79(s, 9H), 1.59(d, 7.2 Hz, 3H) |
| 30 | 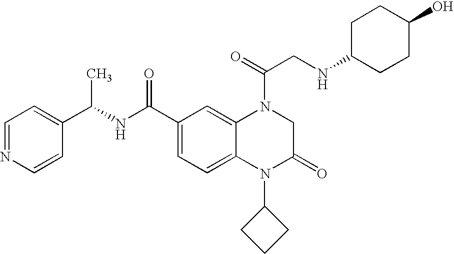 | 2 | 8 | 506 | |
| 31 | 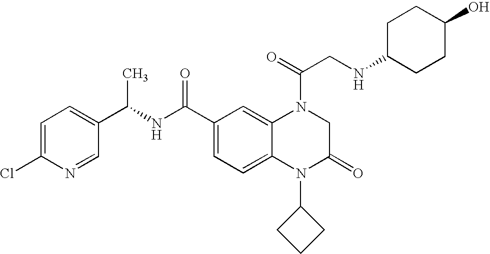 | 2.6 | 8 | 540 | |
| 32 | 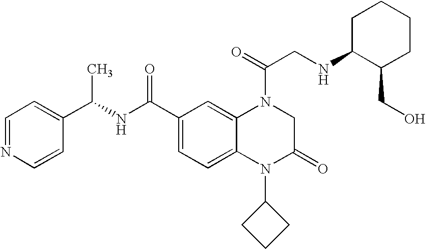 | 2.13 | 8 | 520 | |
| 33 | 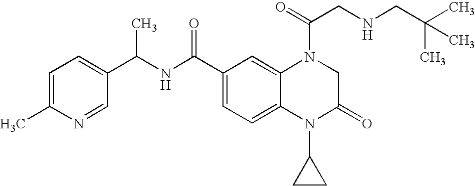 | 1.97 | 8 | 478 | |
| 34 | 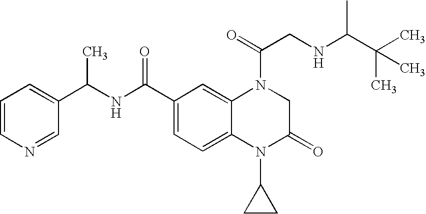 | 3.5 | 1 | 478 | |
| 35 | 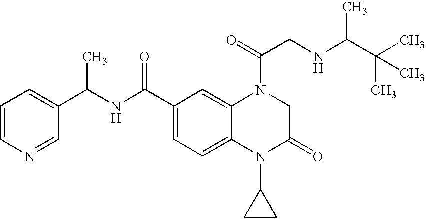 | 3.3 | 1 | 506 | |
| 36 | 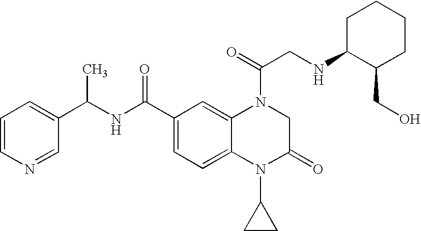 | 2.07 | 8 | 492 | |
| 37 | 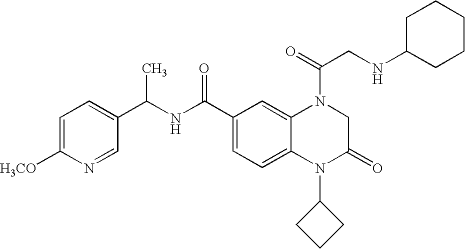 | 3.8 | 1 | 520 | |
| 38 | 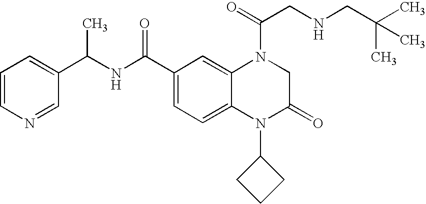 | 3.7 | 1 | 478 | |
| 39 | 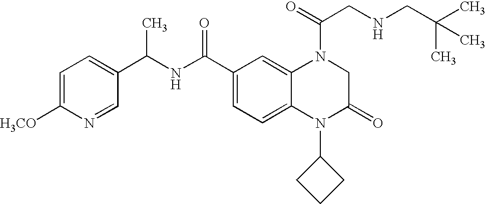 | 3.8 | 1 | 508 | |
| 40 | 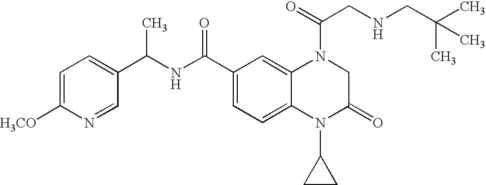 | 3.6 | 1 | 494 | |
| 41 | 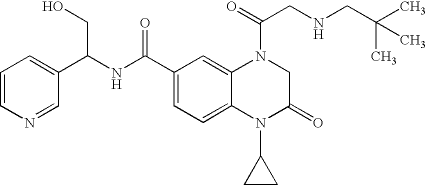 | 3.3 | 1 | 480 | |
| 42 | 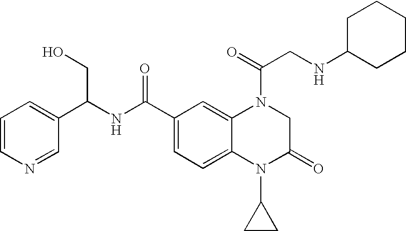 | 3.3 | 1 | 492 | |
| 43 | 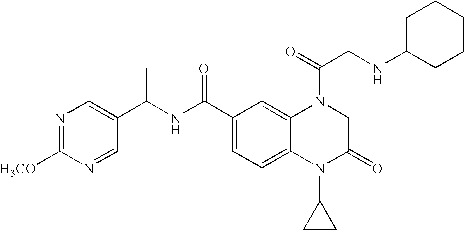 | 2.75 | 8 | 507 | |
| 44 | 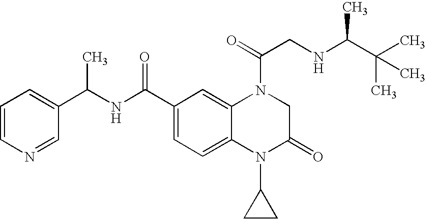 | 3.51 | 1 | 478 | |
| 45 | 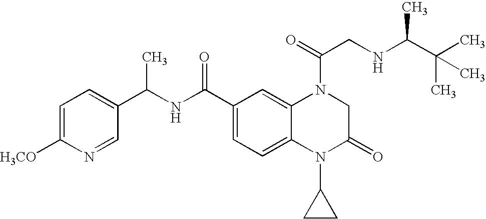 | 3.6 | 1 | 508 | |
| 46 | 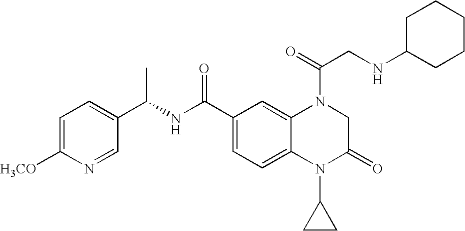 | 3.8 | 1 | 520 | |
| 47 | 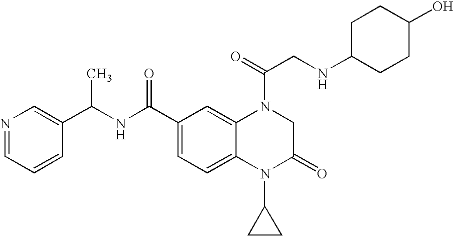 | 3.4 | 1 | 492 | ¹H-NMR(400 MHz, DMSO-d₆): d=0.48-0.60(m, 2H), 0.83-1.22(m, 5H), 1.48(d, 3H), 1.52. 1.70(m, 5H), 2.18-2.30(m, 1H), 2.80-2.88(m, 2H), 3.50 (br. s, 2H), 3.55(m, 1H), 4.42(s, 2H), 5.13(quintet, 1H), 7.37(d, 2H), 7.52(4, 8.07(s, 1H), 8.48(d, 1H), 8.86(d, 1H) |
| 48 | 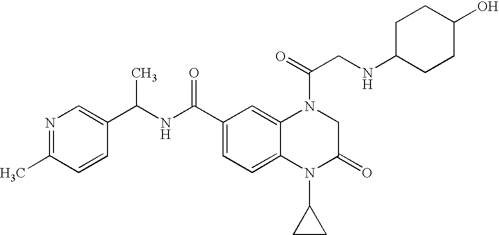 | 3.6 | 1 | 506 | ¹H-NMR(400 MHz, DMSO-d₆): =0.48-0.60(m, 2H), 0.80-1.21(m, 5H), 1.48(d, 3H), 1.52-1.70(m, 5H), 2.18-2.30(m, 1H),2.33(s, 3H), 2.80-2.88(m, 2H), 3.50(br. s, 2H), 3.55(m, 1H), 4.42(s, 2H), 5.13(quintet, 1H), 7.37(d, 2H), 7.52(d, 1H), 7.88(d, 1H), 8.07(br. s, 1H), 8.50(d, 2H), 8.86(d, 1H) |
| 49 | 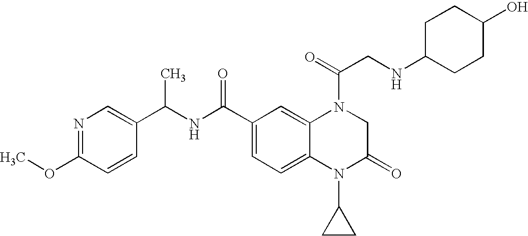 | 3.8 | 1 | 522 | ¹H-NMR(400 MHz, DMSO-d₆): =0.48-0.60(m, 2H), 0.80-1.21(m, 5H), 1.48(d, 3H), 1.52-1.70(m, 5H), 2.18-2.30(m, 1H), 2.55(s, 3H), 2.80-2.88(m, 2H), 3.50(br. s, 2H), 3.55(m, 1H), 4.42(s, 2H), 5.13(quintet, 1H), 7.37(d, 2H), 7.52(d, 1H), 7.88(d, 1H), 8.07(br. s, 1H), 8.50(d, 2H), 8.86(d, 1H) |
| 50 | 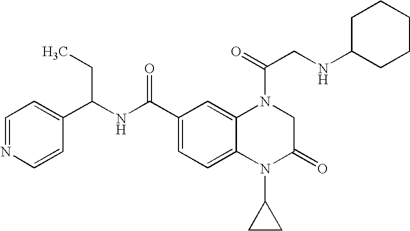 | 3.5 | 1 | 490 | |
| 51 | 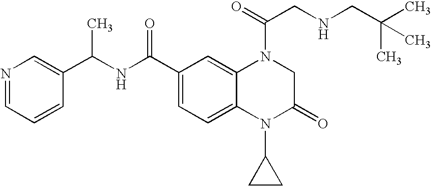 | 3.6 | 1 | 464 | |
| 52 | 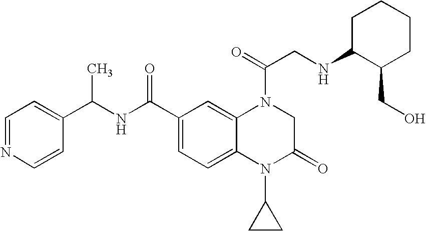 | 3.3 | 1 | 492 | |
| 53 | 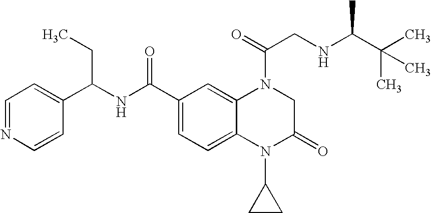 | 3.6 | 1 | 492 | |
| 54 | 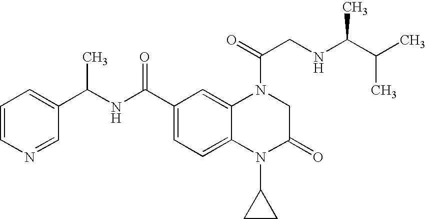 | 3.5 | 1 | 492 | |
| 55 | 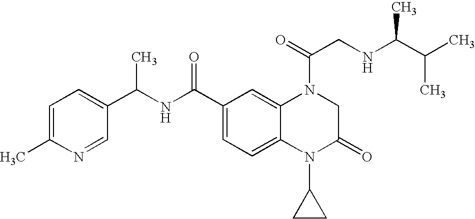 | 3.6 | 1 | 492 | |
| 56 | 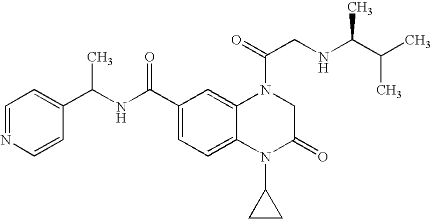 | 3.4 | 1 | 492 | |

### B. ASSESSMENT OF THE PHYSIOLOGICAL ACTIVITY

Abbreviations:

DMEM Dulbecco's modified Eagle medium
FCS fetal calf serum
HEPES 4-(2-hydroxyethyl)-1-piperazineethanesulphonic acid

1. in vitro Tests to Determine the M2 Activity and Selectivity

a) Cellular Functional in vitro Test

A recombinant cell line was used to identify agonists of the human M2 acetylcholine receptor (M2A ChR) and to quantify the activity of the substances described herein. The cell is originally derived from a hamster ovary epithelial cell (Chinese Hamster Ovary, CHO K1, ATCC: American Type Culture Collection, Manassas, Va. 20108, USA). The test cell line constitutively expresses a modified form of the calcium-sensitive photoprotein aequorin which after reconstitution with the cofactor coelenterazine emits light when the free calcium concentration in the inner mitochondrial compartment is increased (Rizzuto R. Simpson A W, Brini M, Pozzan T.; Nature 358 (1992) 325-327). The cell is additionally stably transfected with the human M2AChR (Peralta E G, Ashkenazi A, Winslow J W, Smith D H, Ramachandran J, Capon, D J, EMBO Journal 6 (1987) 3923-3929) and with the gene which codes for the promiscuous G_{α16} protein (Amatruda T T, Steele D A, Slepak V Z, Simon M I, Proceedings in the National Academy of Science USA 88 (1991), 5587-5591). The resulting M2AChR test cell responds to stimulation of the recombinant M2ACh receptor with an intracellular release of calcium ions, which can be quantified through the resulting aequorin luminescence with a suitable luminometer (Milligan G, Marshall F, Rees S, Trends in Pharmacological Sciences 17 (1996) 235-237).

The in vitro selectivity for the muscarinergic acetylcholine receptor subtypes M1 to M5 is determined by using appropriate CHO K1 cells which are stably transfected likewise with the gene of the calcium-sensitive photoprotein aequorin and the gene of the M1, M3 or M5 receptor subtypes or, in the case of the M4 receptor subtypes, additionally with the gene of the promiscuous G_{α16} protein.

Test procedure: The cells are plated out on the day before the test in culture medium (DMEM, 10% FCS, 2 mM glutamine, 10 mM HEPES; Gibco Cat.#21331-020; now belongs to: Invitrogen GmbH, 76131 Karlsruhe) in 384 (or 1536) well microtiter plates and kept in a cell incubator (96% humidity, 5% v/v CO₂, 37° C.). On the day of the test, the culture medium is replaced by a Tyrode solution (in mM: 140 NaCl, 5 KCl, 1 MgCl₂, 2 CaCl₂, 20 glucose, 20 HEPES) which additionally contains the cofactor coelenterazine (50 μM), and the microtiter plate is then incubated for a further 3-4 hours. Immediately after the test substances have been transferred into the wells of the microtiter plate, the resulting light signal is measured in the luminometer. The results are shown in Table A:

TABLE A

| Example No. | EC₅₀ (nM) |
|---|---|
| 2 | 1000 |
| 12 | 1800 |
| 7 | 5 |
| 11 | 320 |
| 5 | 37 |
| 22 | 150 |
| 23 | 18 |
| 24 | 32 |
| 25 | 6 |
| 26 | 16 |

b) Binding Studies on Human Muscarinergic Acetylcholine Receptors

Stably transfected CHO K1 cells which express the human muscarinergic M2 receptor are, after 80% confluence is reached, suspended in 10 ml of binding buffer (20 mM 40(2-hydroxyethyl)-1-piperazineethanesulphonic acid, 5 mM in magnesium chloride, pH 7.4) per 175 cm² cell culture bottle and homogenised using an Ultra-Turrax apparatus. The homogenates are centrifuged at 1000 g and 4° C. for 10 minutes. The supernatant is removed and centrifuged at 20 000 g and 4° C. for 30 min. The membrane sediment with the M2 receptors is taken up in 10 ml of binding buffer and stored at −70° C.

For the binding test, 2 nM ³H-oxotremorine M (3200 GBq/mmol, Perkin Elmer) are incubated with 100-1000 μg/ml M2 membranes per mixture (0.2 ml) in the presence of the test substances at room temperature for 60 minutes. The incubation is stopped by centrifugation at 10 000 g for 10 minutes and subsequent washing wit 0.1% bovine serum albumin in binding buffer at 4° C. Centrifugation is again carried out at 10 000×g and 4° C. for 10 minutes. The sediment is resuspended in 0.1 ml of 1 N sodium hydroxide solution and transferred into scintillation vials. After addition of 4 ml of Ultima Gold scintillator, the radioactivity bound to the membranes is quantified using a BeckinanCoulter LS6000 IC scintillation counter. The nonspecific binding is defined as radioactivity in the presence of 10 μM oxotremorine M and is usually less than 5% of the bound total radioactivity. The binding data (IC₅₀ and dissociation constant Ki) are determined using the graph pad prism version 3.02 programme.

2. in vivo Test to Detect the Cardiovascular Effect

a) Langendorff Guinea Pig Heart

The heart is removed from anaesthetised guinea pigs after opening the thoracic cavity and introduced into a conventional Langendorff apparatus. The coronary arteries are perfused at constant volume (10 ml/min) and the profusion pressure arising during this is recorded via an appropriate pressure transducer. A decrease in the profusion pressure in this arrangement corresponds to a relaxation of the coronary arteries. At the same time, the pressure developed by the heart during each contraction is measured via a balloon inserted into the left ventricle and a further pressure transducer. The rate of the heart beating in isolation is found by calculation from the number of contractions per unit time.

b) Blood Pressure Measurements on Anaesthetised Rats

Male Wistar rats with a body weight of 300-350 g are anaesthetised with thiopental (100 mg/kg i.p.). After tracheotomy, a catheter is introduced into the femoral artery to measure the blood pressure. The substances to be tested are administered orally in Transcutol, Cremophor EL, H₂O (10%/20%/70%) in a volume of 1 ml/kg.

c) Effect on the Mean Blood Pressure of Conscious Spontaneously Hypertensive Rats

Continuous blood pressure measurements over 24 hours are carried out on spontaneously hypertensive female rats (MOL:SPRD) weighing 200-250 g and moving freely. For this purpose, pressure transducers (Data Sciences Inc., St. Paul, Minn., USA) are implanted chronically in the descending abdominal aorta below the renal artery of the animals, and the transmitter connected thereto is fixed in the abdominal cavity. The animals are kept singly in type III cages which are positioned on the individual receiving stations and are adapted to a 12-hour light/dark rhythm. Water and feed are freely available. For data acquisition, the blood pressure of each rat is recorded for 10 seconds every 5 minutes. The measurements are combined in each case for a period of 15 minutes and the mean is calculated from these values. The test compounds are dissolved in Transcutol (10%), Cremophor (20%), H₂O (70%) mixture and administered orally by gavage in a volume of 2 ml/kg of body weight. The test doses are between 0.3-30 mg/kg of body weight.

d) Blood Pressure and Heart Rate Measurements on Anaesthetised Dogs

The experiments are carried out on dogs (mongrel) of both sexes with a body weight between 20 and 30 kg. Anaesthesia is induced by a slow i.v. injection of 25 mg/kg thiopental (Trapanal®) and continued during the experiment by continuous infusion of 0.08 mg/kg/h fentanyl (Fentanyl®) and 0.25 mg/kg/h droperidol (Dehydrobenzperidol®). Alloferin (0.02 mg/kg/h) is added as muscle relaxant. The dogs are artificially ventillated with 1 part of nitrous oxide and 3 parts of oxygen. The test substances are administered intravenously via the femoral vein.

A MillarTip catheter is passed via the carotid artery into the left ventricle to pick up the left ventricular pressure and calculate the contractility. A hollow catheter is introduced via the femoral artery into the aorta and connected to a pressure transducer to measure the peripheral blood pressure. After a left-sided thoracotomy, the left circumflex (LCX) or the left anterior descending (LAD) coronary artery is exposed and an electromagnetic flow head is sited to measure the coronary flow. The ECG is recorded via an extremity lead and an ECG amplifier, and the heart rate and ECG parameters are found from the recorded ECG. The oxygen saturation at the coronary sinus is determined via a Swan-Gantz oximetry TD catheter.

### B. Exemplary Embodiments of Pharmaceutical Compositions

The compounds of the invention can be converted into pharmaceutical preparations in the following ways:

Tablet:

Composition:

100 mg of the compound of Example 1, 50 mg of lactose (monohydrate), 50 mg of maize starch (native), 10 mg of polyvinylpyrolidone (PVP 25) (from BASF, Ludwigshafen, Germany) and 2 mg of magnesium stearate.

Tablet weight 212 mg. Diameter 8 mm, radius of curvature 12 mm.

Production:

The mixture of active ingredient, lactose and starch is granulated with a 5% strength solution (m/m) of the PVP in water. The granules are dried and then mixed with the magnesium stearate for 5 min. This mixture is compressed using a conventional tablet press (see above for format of the tablet). A compressive force of 15 kN is used as guideline for the compression.

Suspension Which Can Be Administered Orally:

Composition:

1000 mg of the compound of Example 1, 1000 mg of ethanol (96%), 400 mg of Rhodigel (xanthan gum from FMC, Pennsylvania, USA) and 99 g of water.

10 ml of oral suspension correspond to a single dose of 100 mg of the compound of the invention.

Production:

The Rhodigel is suspended in ethanol, and the active ingredient is added to the suspension. The water is added while stirring. The mixture is stirred for about 6 h until the swelling of the Rhodigel is complete.

1. A compound of the formula 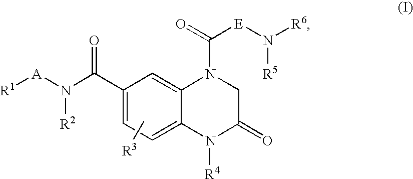
in which
A is a C₁-C₆-alkanediyl chain which is optionally substituted by one or two hydroxy groups,
E is a C₁-C₆-alkanediyl chain,
R¹ is heteroaryl, where heteroaryl is optionally substituted by 1 to 3 substituents independently of one another selected from the group consisting of halogen, hydroxy, amino, trifluoromethyl, nitro, cyano, alkyl, alkoxy, alkylamino, alkoxycarbonyl, aminocarbonyl and alkylaminocarbonyl,
R² is hydrogen, alkyl or cycloalkyl,
R³ is hydrogen, halogen, alkyl or alkoxy,
R⁴ is alkyl or cycloalkyl, where alkyl and cycloalkyl are optionally substituted by 1 to 3 substituents independently of one another selected from the group consisting of halogen, hydroxy, amino, alkyl, alkoxy, alkylamino, alkoxycarbonyl, aminocarbonyl and alkylaminocarbonyl,
R⁵ is hydrogen, alkyl or cycloalkyl, and
R⁶ is alkyl, cycloalkyl, heterocyclyl, aryl or heteroaryl, where alkyl, cycloalkyl, heterocyclyl, aryl or heteroaryl are optionally substituted by 1 to 3 substituents independently of one another selected from the group consisting of halogen, hydroxy, amino, trifluoromethyl, nitro, cyano, alkyl which may in turn be substituted by one or two hydroxy groups, or alkoxy, alkylamino, alkoxycarbonyl, aminocarbonyl and alkylaminocarbonyl, or
R⁵ and R⁶ form together with the nitrogen atom to which they are bonded a nitrogen-containing heterocyclyl ring, where heterocyclyl is optionally substituted by 1 to 3 substituents independently of one another selected from the group consisting of halogen, hydroxy, amino, trifluoromethyl, nitro, cyano, alkyl, alkoxy, alkylamino, alkoxycarbonyl, aminocarbonyl and alkylaminocarbonyl,
or a salt, solvate, or solvate of the salt thereof.

2. The compound of the formula (I) according to claim 1, in which
A is a C₁-C₆-alkanediyl chain,
E is a C₁-C₆-alkanediyl chain,
R¹ is heteroaryl, where heteroaryl is optionally substituted by 1 to 3 substituents independently of one another selected from the group consisting of halogen, hydroxy, amino, trifluoromethyl, nitro, cyano, alkyl, alkoxy, alkylamino, alkoxycarbonyl, aminocarbonyl and alkylaminocarbonyl,
R² is hydrogen,
R³ is hydrogen,
R⁴ is alkyl or cycloalkyl, where alkyl and cycloalkyl are optionally substituted by 1 to 3 substituents independently of one another selected from the group consisting of alkyl, alkoxy and alkylamino,
R⁵ is hydrogen, and
R⁶ is alkyl, cycloalkyl, heterocyclyl, aryl or heteroaryl, where alkyl, cycloalkyl, heterocyclyl, aryl or heteroaryl are optionally substituted by 1 to 3 substituents independently of one another selected from the group consisting of halogen, hydroxy, amino, trifluoromethyl, nitro, cyano, alkyl, alkoxy, alkylamino, alkoxycarbonyl, aminocarbonyl and alkylaminocarbonyl,
or a salt solvate or solvate of the salt thereof.

3. The compound of the formula (I) according to claim 1, in which
A is methylene or ethane-1,1-diyl,
E is methylene,
R¹ is 5- or 6-membered heteroaryl, where heteroaryl is optionally substituted by 1 to 3 substituents independently of one another selected from the group consisting of halogen, hydroxy, amino, alkyl, alkoxy, alkylamino, alkoxycarbonyl, aminocarbonyl and alkylaminocarbonyl,
R² is hydrogen,
R³ is hydrogen,
R⁴ is cycloalkyl, where cycloalkyl is optionally substituted by 1 or 2 alkyl substituents,
R⁵ is hydrogen, and
R⁶ is alkyl or cycloalkyl, where cycloalkyl is optionally substituted by 1 to 3 substituents independently of one another selected from the group consisting of alkyl, alkoxy, alkylamino, alkoxycarbonyl, aminocarbonyl and alkylaminocarbonyl,
or a salt, solvate, or solvate of the salt thereof.

4. The compound of the formula (I) according to claim 1, in which
A is ethane-1,1-diyl,
E is methylene,
R¹ is 6-membered heteroaryl, where heteroaryl is optionally substituted by 1 or 2 substituents independently of one another selected from the group consisting of methyl, ethyl, methoxy and ethoxy,
R² is hydrogen,
R³ is hydrogen,
R⁴ is cyclopropyl,
R⁵ is hydrogen, and
R⁶ is cyclohexyl or cyclopentyl, where cyclohexyl or cyclopentyl are optionally substituted by 1 or 2 substituents independently of one another selected from the group consisting of methyl, ethyl, methoxy and ethoxy,
or a salt, solvate, or solvate of the salt thereof.

5. Process for preparing the compounds of the formula (I) as defined in claim 1, characterized in that the compounds of the formula 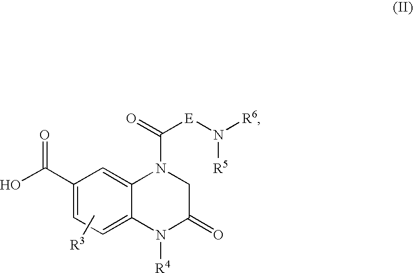
in which
E, R³, R⁴, R⁵ and R⁶ have the meaning stated in claim 1, are reacted with compounds of the formula 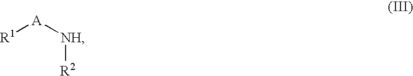
in which
A, R¹ and R² have the meaning stated in claim 1.

6. (canceled)

7. Medicament comprising at least one compound of the formula (I) as claimed in claim 1, and at least one further excipient.

8. Medicament comprising at least one compound of the formula (I) as defined in claim 1, and at least one further active ingredient.

9. A method for treating cardiovascular disorders, comprising administering to a patient in need thereof a therapeutically effective amount of a compound of claim 1.
